SEQUENCE LISTING (10 sequences)

SEQ ID NO: 1 - protein, 436 aa, Staphylococcus aureus [sequence omitted]

SEQ ID NO: 2 - protein, 48 aa, Staphylococcus aureus
ERQNIENADKAIKDFQDNKAPHDKSAAYEANSKLPKDLRDKNNRFVEK

SEQ ID NO: 3 - protein, 69 aa, Staphylococcus aureus
VSIEKAIVRHDERVKSANDAISKLNEKDSIENRRLAQREVNKAPMDVKEHLQKQLDALVAQKDAEKKVA

SEQ ID NO: 4 - protein, 117 aa, Staphylococcus aureus
ERQNIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLDALVAQKDAEKKVA

SEQ ID NO: 5 - protein, 117 aa, Artificial Sequence
  Modified wild type Sbi-III and Sbi-IV
ERQNIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLDALVAQKDAEKKCA

SEQ ID NO: 6 - protein, 24 aa, Artificial Sequence
  Ag85b
GTSGGGGSHHHHHHHHHHSGGGGS

SEQ ID NO: 7 - protein, 129 aa, Artificial Sequence
  Sbi-III and Sbi-IV Test Protein
MRGSHHHHHHGSERQNIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLDALVAQKDAEKKVA

SEQ ID NO: 8 - protein, 322 aa, Artificial Sequence
  Ag85b Test Protein
MPLLLLLPLLWAGALAMDVDKLHHHHHHHHHTSASFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDRSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGKP

SEQ ID NO: 9 - protein, 452 aa, Artificial Sequence
  Sbi-III and Sbi-IV Ag85b fusion test protein
MPLLLLLPLLWAGALAMDVDERQNIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLDALVAQKDAEKKVAGTSGGGGSHHHHHHHHHHSGGGGSTSASFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDRSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGKP

SEQ ID NO: 10 - protein, 129 aa, Artificial Sequence
  Sbi-III and Sbi-IV (V80C) Test protein
MRGSHHHHHHGSERQNIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLDALVAQKDAEKKCA

(19) United States
(12) Patent Application Publication
(10) Pub. No.: US 2019/0282683 A1
(43) Pub. Date: Sep. 19, 2019

(54) IMMUNOGENIC COMPOSITIONS COMPRISING SBI PROTEIN AND USES THEREOF

(71) Applicant: THE UNIVERSITY OF BATH, Bath and East Somerset (GB)

(72) Inventors: Jean VAN DEN ELSEN, Bath and North East Somerset (GB); Andrew Graham WATTS, Bath and North East Somerset (GB); Kevin James MARCHBANK, Newcastle Upon Tyne, Northumberland (GB)

(73) Assignee: THE UNIVERSITY OF BATH, Bath and North East Somerset (GB)

(21) Appl. No.: 16/463,665

(22) PCT Filed: Nov. 24, 2017
(86) PCT No.: PCT/EP2017/080321

§ 371 Date: May 23, 2019

(30) Foreign Application Priority Data
Nov. 25, 2016 (GB) 1619965.5

(51) Int. Cl. A61K 39/09; A61K 39/385; A61K 39/39; A61K 39/04; A61P 31/06

(52) U.S. Cl. CPC A61K 39/092; A61K 39/385; A61K 2039/55516; A61K 39/04; A61P 31/06; A61K 39/39

(57) ABSTRACT

The invention relates to methods and compositions for use in stimulating an immune response against a target antigen. More specifically, it relates to use of domains III and IV of the Staphylococcal Sbi protein as an immunological adjuvant, for enhancing an immune response against a target antigen.

### FIELD OF THE INVENTION

The invention relates to methods and compositions for use in stimulating an immune response against a target antigen. More specifically, it relates to use of domains III and IV of the Staphylococcal Sbi protein as an immunological adjuvant, for enhancing an immune response against a co-administered target antigen.

### BACKGROUND TO THE INVENTION

Many bacterial pathogens have evolved ways to adapt to their host environment and to survive host immune system attack by producing a variety of immuno-modulating factors.

Gram-positive human pathogen Staphylococcus aureus has a vast arsenal of intrinsic factors that can regulate both adaptive and innate immune systems in a variety of hosts and in addition has evolved elements that enable the bacterium to hijack host immuno-regulators enabling it to persist in the host environment.

Currently six intrinsic complement modulators secreted by Staphylococcus aureus have been identified and characterized. They include Staphylococcal complement inhibitor (SCIN) which binds to the classical (C4b2a) and alternative (C3bBb) pathway C3 convertases at a bacterial surface, stabilizing them and inhibiting their enzymatic activity. The C-terminal fragment of extracellular fibrinogen-binding protein EFb-C and its homologue Ehp bind to the C3d region of C3, the central complement component, inhibiting C3b deposition on target surfaces. Staphylococcal superantigen-like protein 7 (SSL7) affects the terminal pathway by binding to C5 and in so doing inhibits the complement-mediated bactericidal activity of human serum, most likely by preventing C5 cleavage by C5 convertases. Chemotaxis inhibitory protein of S. aureus (CHIPS) binds to the C5a receptor presented on phagocytes in a way that prevents signaling via the inflammatory anaphylatoxin C5a.

“S. aureus binder of immunoglobulin” (Sbi), the most recently characterised of the S. aureus immuno-modulators, affects the adaptive immune system by sequestering host IgG through the formation of insoluble complexes (Atkins et al., 2008, Mol Immunol 45, 1600-1611). In addition to immunoglobulin binding domains I and II, Sbi contains two further domains (Sbi-III and IV) that can bind C3d (in native C3, iC3b and C3dg) and in concert inhibit the alternative pathway by causing futile fluid phase consumption of C3, the most abundant complement component (Burman et al., 2008, J Biol Chem 283, 17579-17593), via a covalent adduct with activated C3b.

WO2007/138328 proposes use of constructs containing the Sbi-III and IV domains to down-regulate inflammatory immune responses by consuming complement components.

### SUMMARY OF THE INVENTION

The invention makes use of the ability of Sbi domains III and IV, acting in concert, to activate the key complement protein C3.

S. aureus employs this activity to inhibit the host's innate immune system by depleting C3 and so preventing effective activation of the complement pathway.

However, the present inventors have found that co-administration of Sbi-III-IV with a target antigen can in fact enhance the immune reaction against that target antigen. Thus, in effect, Sbi-III-IV is capable of acting as an adjuvant. Without wishing to be bound by any particular theory, it is believed that the presence of Sbi-III-IV causes local activation of C3, which may result in opsonisation of the target antigen (e.g. by C3 breakdown products such as C3b and C3d), consequently enhancing the immune response generated against the target antigen.

Thus, in its broadest form, the invention provides a complement-activating moiety comprising Sbi-III-IV for use as an immunological adjuvant.

Typically, use as an immunological adjuvant entails administration of the complement-activating moiety to a subject in conjunction with a target antigen, for enhancing an immune response in the subject against that target antigen.

Thus, the invention provides a complement-activating moiety comprising Sbi-III-IV for use in a method of immune stimulation, wherein the method comprises administering said complement-activating moiety to a subject in conjunction with a target antigen, for enhancing an immune response in the subject against the target antigen.

This may alternatively be described as providing a complement-activating moiety comprising Sbi-III-IV for use in a method of enhancing an immune response in a subject against a target antigen, wherein said complement-activating moiety is administered to a subject in conjunction with the target antigen.

The invention also provides a complement-activating moiety comprising Sbi-III-IV for use in a method of stimulating an immune response against a target antigen, wherein the method comprises administering said complement-activating moiety to a subject in conjunction with the target antigen, wherein the complement-activating moiety enhances an immune response in the subject against the target antigen.

The invention further provides a method of immune stimulation, comprising administering a complement-activating moiety comprising Sbi-III-IV as an immunological adjuvant.

The invention further provides a method of immune stimulation, wherein the method comprises administering a complement-activating moiety comprising Sbi-III-IV to a subject in conjunction with a target antigen, for enhancing an immune response in the subject against the target antigen.

The invention further provides a method of enhancing an immune response in a subject against a target antigen, wherein said method comprises administering a complement-activating moiety comprising Sbi-III-IV to the subject in conjunction with the target antigen.

The invention also provides a method of stimulating an immune response against a target antigen, wherein the method comprises administering a complement-activating moiety comprising Sbi-III-IV to a subject in conjunction with the target antigen, wherein the complement-activating moiety enhances an immune response in the subject against the target antigen.

The invention further provides relates to the use of a complement-activating moiety comprising Sbi-III-IV in the preparation of a medicament for use as an immunological adjuvant.

The invention further provides the use of a complement-activating moiety comprising Sbi-III-IV in the preparation of a medicament for use in a method of immune stimulation, wherein the method comprises administering said complement-activating moiety to a subject in conjunction with a target antigen for enhancing an immune response in the subject against the target antigen.

The invention further provides the use of a complement-activating moiety comprising Sbi-III-IV in the preparation of a medicament for use in a method of enhancing an immune response in a subject against a target antigen, wherein said method comprises administering the complement-activating moiety to the subject in conjunction with the target antigen.

The invention also provides the use of a complement-activating moiety comprising Sbi-III-IV in the preparation of a medicament for use in a method of stimulating an immune response against a target antigen, wherein the method comprises administering said complement-activating moiety to a subject in conjunction with the target antigen, wherein the complement-activating moiety enhances the immune response in the subject against the target antigen.

In any of the aspects described above, the complement-activating moiety and the target antigen may be provided in the same composition (e.g. in admixture) or in separate compositions. It will typically be desirable that they are provided in the same composition. However, depending on the specific components, this may not be possible, for example if they have incompatible requirements for formulation. If the complement-activating moiety and the target antigen are provided in separate compositions, they will typically be administered at substantially the same site, by the same route, and within one hour of one another, more preferably within 30 minutes, within 15 minutes, within 5 minutes or within 1 minute of one another, e.g. substantially simultaneously. The composition or compositions may comprise a carrier, e.g. a pharmaceutically acceptable carrier.

In an alternative approach, the target antigen may be opsonised in vitro or ex vivo before being administered to the recipient subject.

Thus the invention further provides a method of enhancing the immunogenicity of a target antigen, comprising contacting the target antigen in vitro or ex vivo with complement and a complement-activating moiety comprising Sbi-III-IV to yield an opsonised target antigen.

Once again, it is believed that activation of complement by Sbi-III-IV results in opsonisation of the target antigen. When the opsonised target antigen is administered to a subject, the subject's immune response against the target antigen will typically be enhanced as compared to the immune response which would be stimulated by an equivalent administration of an identical target antigen which has not been subjected to such in vitro or ex vivo opsonisation.

Thus the method may comprise the subsequent step of administering the target antigen to a subject.

Before administration, the opsonised target antigen may be isolated from other components of the opsonisation mixture. For example, the opsonised target antigen may be substantially isolated from other complement components before administration. Additionally or alternatively, the opsonised target antigen may be substantially isolated from the complement-activating moiety, especially when the target antigen and complement-activating moiety are not covalently linked.

Whether or not any such isolation is performed, the method may further comprise the step of formulating the opsonised target antigen for administration to a subject, e.g. as a pharmaceutical composition.

The invention also provides a method of stimulating an immune response against a target antigen, wherein the method comprises administering to the subject a target antigen which has previously been contacted in vitro or ex vivo with complement and a complement-activating moiety comprising Sbi-III-IV.

The invention further provides a composition, e.g. a pharmaceutical composition, comprising a target antigen which has been opsonised by contact in vitro or ex vivo with complement and a complement-activating moiety comprising Sbi-III-Iv.

The invention also provides a composition comprising an opsonised target antigen for use in a method of stimulating an immune response against the target antigen, wherein the target antigen has previously been contacted in vitro or ex vivo with complement and a complement-activating moiety comprising Sbi-III-IV.

The invention also provides the use of a composition comprising a target antigen in the preparation of a medicament for stimulating an immune response against the target antigen, wherein the target antigen has previously been contacted in vitro or ex vivo with complement and a complement-activating moiety comprising Sbi-III-IV.

The opsonisation may be achieved by admixing the target antigen and complement-activating moiety in vitro or ex vivo with a system comprising complement.

The system may comprise whole blood, plasma, serum, or a fraction thereof. The whole blood, plasma or serum may be derived from the same species as the intended recipient subject, from the subject themselves, or from an individual syngeneic with the subject. This reduces the risk of any unwanted immune response against immunogenically foreign complement components.

Alternatively, the system may be assembled in vitro, e.g. from isolated (e.g. recombinant) complement proteins and/or cells capable of expressing and secreting complement proteins.

The system will typically comprise at least C3 protein. It may also comprise at least Factor I, and one or more of Factor H, soluble complement receptor 1 (sCR1) and C4 binding protein (C4BP). Other complement components may be added as desired to achieve the desired opsonisation of the target antigen following complement activation by the complement-activating moiety.

Again, the complement components may be obtained from the same species as the intended recipient subject, from the subject themselves, or from an individual syngeneic with the subject, or they may be recombinant forms thereof.

Opsonisation in vitro or ex vivo may be particularly useful when the target antigen and complement-activating moiety are not physically linked, but can be used with any arrangement of these components.

In all aspects of the invention, the target antigen may be any suitable molecule against which it is desirable to raise an immune response, and especially an antibody response.

The antigen may be a peptide antigen. The term “peptide” refers to the nature of the antigen, i.e. that it is formed from amino acids linked by peptide bonds, and should not be taken to imply any particular size or length. Typically the peptide antigen will be at least 8, at least 9, at least 10, at least 11, at least 12, at least 13 or at least 14 amino acids in length, and may be up to 10 amino acids in length, up to 20 amino acids in length, up to 30 amino acids in length, up to 50 amino acids in length, up to 100 amino acids, up to 200 amino acids, or even longer. The peptide antigen may consist of a peptide suitable for presentation by a MHC class I or class II molecule, or may comprise the sequence of a peptide suitable for presentation by a MHC class I or class II molecule and be capable of being processed intracellularly to yield such a peptide. Peptides presented via MHC class I molecules are typically 8 or 9 amino acids in length, while peptides presented via MHC class II molecules are typically 14 to 20 amino acids in length.

Alternatively, the antigen may be, or may comprise, any other biological molecule, including a carbohydrate, polysaccharide, lipid, lipopolysaccharide, etc. Examples include a polysaccharide or lipopolysaccharide from the cell membrane, cell wall or capsule of an infectious organism, such as the polysaccharide Pn6B from Streptococcus pneumoniae.

The target antigen may be covalently linked (e.g. chemically conjugated) to the complement-activating moiety.

Where the target antigen is a peptide, it may be provided as part of a fusion protein with the complement-activating moiety; i.e. the antigen and complement-activating moiety are part of the same peptide chain.

Where the target antigen is not a peptide, and is not covalently linked to the complement-activating moiety, it may be covalently linked to a carrier peptide. Again, the term “peptide” is not intended to limit the size of the carrier molecule, but only to indicate its nature.

The carrier peptide will typically be immunogenic in the intended recipient. For example, it may consist of a peptide suitable for presentation by a MHC class I or class II molecule, or may comprise the sequence of a peptide suitable for presentation by a MHC class I or class II molecule and be capable of being processed intracellularly to yield such a peptide, wherein the peptide is immunogenic in the recipient (i.e. the peptide is recognised as immunologically foreign, or “non-self”, by the immune system of the intended recipient). For the avoidance of doubt, the carrier peptide is not a complement-activating moiety as defined in this specification.

Suitable carriers include keyhole limpet hemocyanin (KLH), ovalbumin (OVA), bovine serum albumin (BSA) and fragments thereof capable of being presented by a MHC class I or class II molecule or of being processed to such a peptide. Other suitable carriers will be known to the skilled person. Thus the carrier will typically be at least 8, at least 9, at least 10, at least 11, at least 12, at least 13 or at least 14 amino acids in length, and may be up to 10 amino acids in length, up to 20 amino acids in length, up to 30 amino acids in length, up to 50 amino acids in length, up to 100 amino acids, up to 200 amino acids, or even longer.

The invention further provides a method of enhancing the immunogenicity of a target antigen, comprising associating said target antigen with a complement-activating moiety comprising Sbi-III-IV. The target antigen may be associated with the complement-activating moiety by, for example:

(i) admixing the target antigen with the complement-activating moiety;
(ii) covalently linking the target antigen to the complement-activating moiety; or
(iii) expressing the target antigen as a fusion protein with the complement-activating moiety.

The target antigen may be derived from an infectious organism, such as a bacterium, fungal cell, virus, protozoan, or other parasite.

Thus the methods and compositions of the invention may be employed for the prophylaxis or treatment of infection by the relevant infectious organism.

The target antigen may be a marker expressed specifically or preferentially on a neoplastic cell, e.g. a cancer cell.

Thus the methods and compositions of the invention may be employed for the prophylaxis or treatment of neoplasia, e.g. cancer.

The subject to which the complement-activating moiety and target antigen are to be administered is typically a mammal. For example, the subject may be a primate (e.g. Old World monkey, New World monkey, ape or human), rodent (e.g. mouse or rat), canine (e.g. domestic dog), feline (e.g. domestic cat), equine (e.g. horse), bovine (e.g. cow), caprine (e.g. goat), ovine (e.g. sheep) or lagomorph (e.g. rabbit).

The method may comprise a single administration, or a sequence of two or more administrations separated by suitably-determined intervals of time. The method may comprise a priming step (i.e. a first administration) followed by one or more boosting steps (a subsequent administration or administrations). For example, a first administration (“prime”) and second administration (“boost”) may be separated by one or more days, one or more weeks, or one or more months, preferably between two weeks and one month. Subsequent administrations (further “boost” administrations) may be provided after one or more weeks or months. Both priming and boosting steps require administration of the target antigen. the complement-activating component may be administered only in the priming step, only in the boosting step, or in both priming and boosting steps.

Administration of the complement-activating moiety in conjunction with the target antigen enhances a subject's immune response against the target antigen. “Enhancement” in this context does not require that the subject has pre-existing immunity against the target antigen. Rather, it means that the immune response generated against the target antigen is greater than would have been achieved without the additional complement activation (and consequent immune stimulation) that the complement-activating moiety provides.

For example, the immune response is greater than would have been achieved by an equivalent administration regime without the complement-activating moiety, or with an inactive analogue of the complement-activating moiety.

Enhancement of the immune response may be measured in any appropriate manner, depending on the nature of the desired immune response. For example, the titre of immunoglobulin (e.g. IgG) specific for the target antigen may be increased.

An equivalent administration regime would typically employ the same dose of target antigen, carrier peptide (if any), route of administration, and dosing pattern. The complement-activating moiety may be absent, or replaced by an inactive analogue.

The target antigen may be administered in conjunction with one or more further adjuvants, i.e. in addition to the complement-activating moiety.

Any appropriate adjuvant may be used. For example, the adjuvant may be an agonist for CD40 (such as soluble CD40 ligand or an agonist antibody specific for CD40), an agonist of CD28, CD27 or OX40 (e.g. an agonist antibody specific for one of those molecules), a CTLA-4 antagonist (e.g. a blocking antibody specific for CTLA-4), a Toll-like receptor (TLR) agonist, 5′ triphosphate RNA, a β-glucan such as curdlan (β-1,3-glucan), or a pro-inflammatory cytokine such as TNF-α or IL-1.

A TLR agonist is a substance which activates a Toll-like receptor such as TLR3, TLR4, TLR5, TLR7 or TLR8. Known TLR agonists include MPL (monophosphoryl lipid A), which binds TLR4; LTA (lipoteichoic acid, which binds TLR2; Poly I:C (polyinosine-polycytidylic acid), which binds TLR3; flagellin, which binds TLR5; resiquimod (R-848; 1-[4-amino-2-(ethoxymethyl)imidazo[4,5-c]quinolin-1-yl]-2-methylpropan-2-ol 1-[4-amino-2-(ethoxymethyl)imidazo[4,5-c]quinolin-1-yl]-2-methylpropan-2-ol) or polyU RNA which bind TLR7 in mice and are believed to bind TLR8 in humans; CpG (DNA CpG motifs), which binds TLR9. For more details, see Reis e Sousa, Toll-like receptors and dendritic cells. Seminars in Immunology 16:27, 2004.

The invention will now be described in more detail, by way of example and not limitation, by reference to the accompanying drawings and examples.

### DESCRIPTION OF THE DRAWINGS

FIG. 1: Complement activation assay. Fresh CD21^{-/-} serum was added to Sbi-III-IV-Ag85b or Sbi-III-IV. The reaction was stopped at various time points (0, 30, 60, 120 minutes). Western blot was developed with rabbit anti-C3 at 1/1000 and goat anti-rabbit at 1/2000. C3d is shown as confirmation that C3 has been activated and broken down. (-) is CD21^{-/-} incubated for 120 minutes with saline.

FIG. 2: Experiment 1 (I.P.1). Serum IgG reactivity to Ag85b over time in WT mice when immunised and boosted (day 28) intraperitoneally with either 1 μg Ag85b or 1.35 μg Sbi-III-IV-Ag85b as detected by ELISA. Sera was diluted 1/50 and the mean absorbance ±SEM of each mouse group is shown. All data has been normalised to the day 0 average of all WT mice. tOD450=normalised value read at 450 nm.

FIG. 3: Experiment 2 (I.V.). Serum IgG reactivity to Ag85b over time in WT mice when immunised and boosted (day 28) intravenously with either 1 μg Ag85b or 1.35 μg Sbi-III-IV-Ag85b as detected by ELISA. Sera from days 0-28 was diluted 1/50 and sera from days 35-50 was diluted 1/100 and multiplied by 2. The mean absorbance ±SEM of each mouse group is shown. All data has been normalised to the day 0 average of all WT mice. Note, no blood sample was taken on the boost day 28 due to the IV administration of the vaccine.

FIG. 4: Experiment 3 (I.P.2). Serum IgG reactivity to Ag85b over time in WT mice when immunised and boosted (day 28) intraperitoneally with either 2.7 μg Sbi-III-IV-Ag85b protein, 2 μg Ag85b, or 0.7 μg Sbi-III-IV plus 2 μg Ag85b in 150 mM NaCl solution as detected by ELISA. Sera was diluted 1/50 and the mean absorbance ±SEM of each mouse group is shown. All data has been normalised to the day 0 average of all WT mice.

FIG. 5:

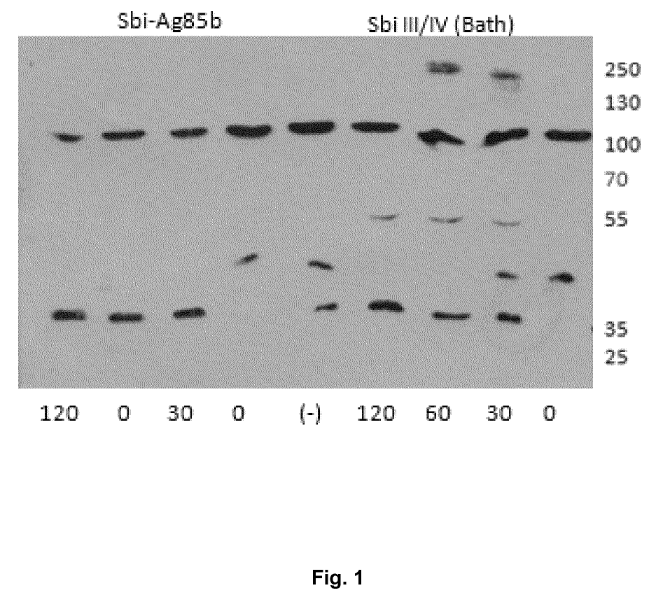

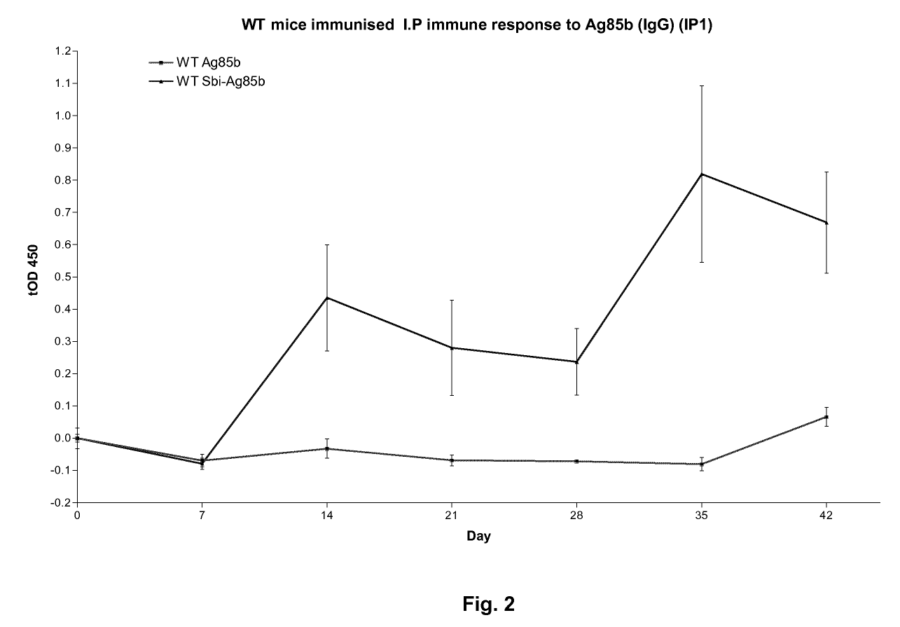

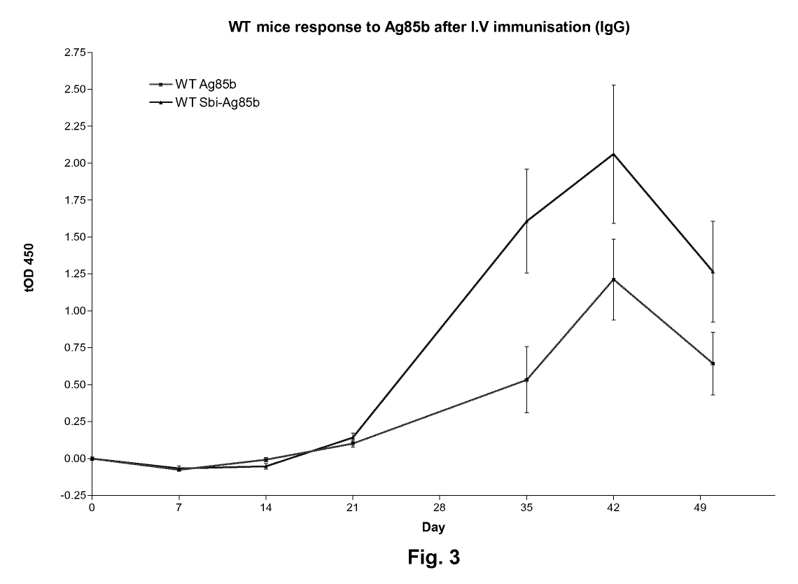

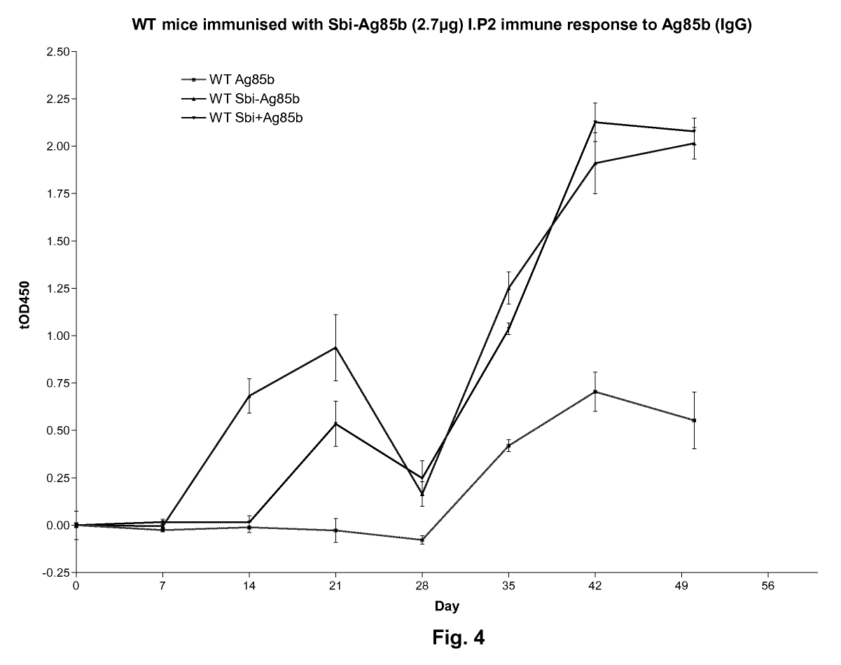

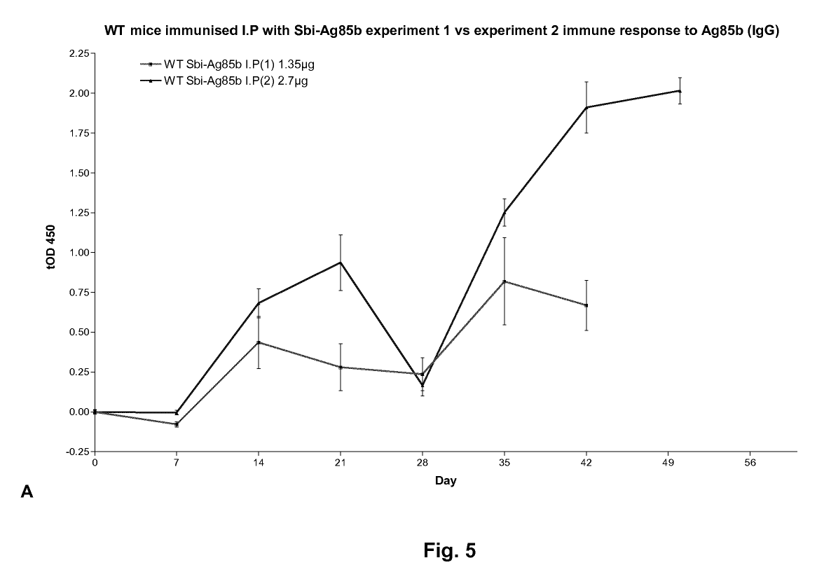

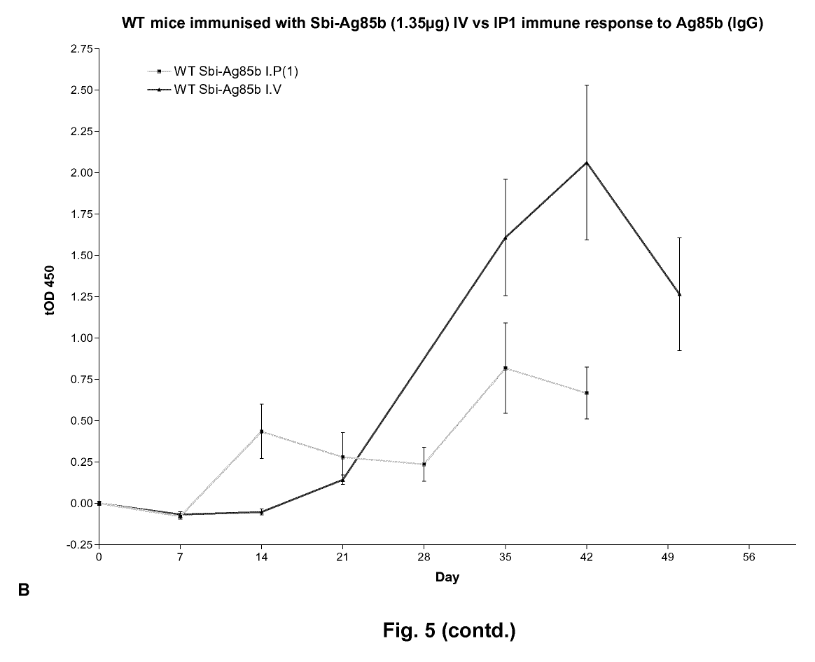

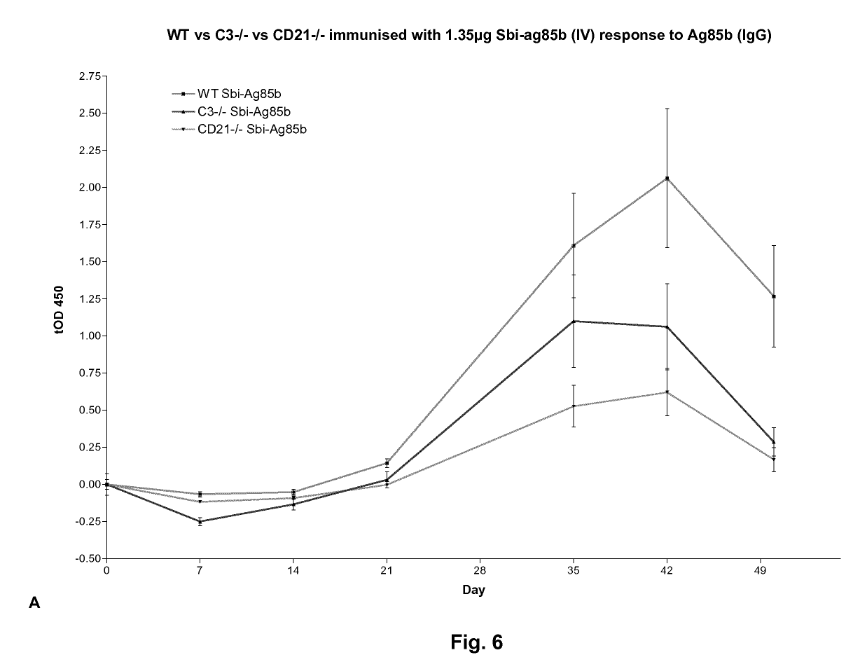

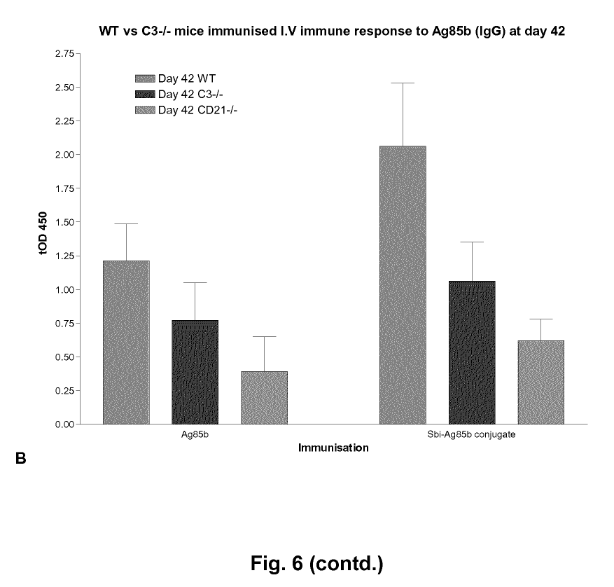

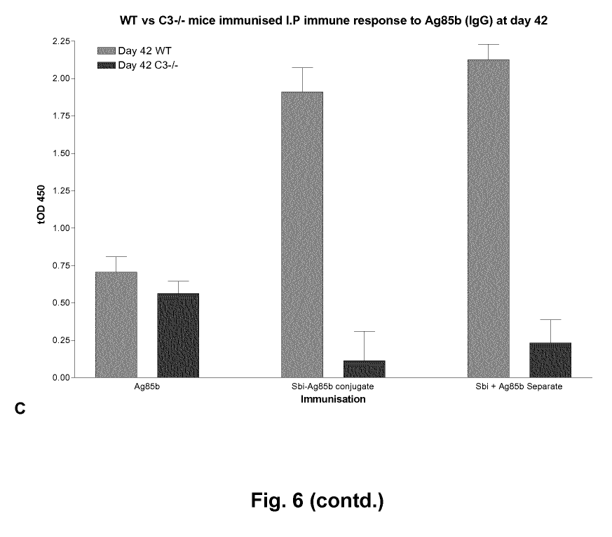

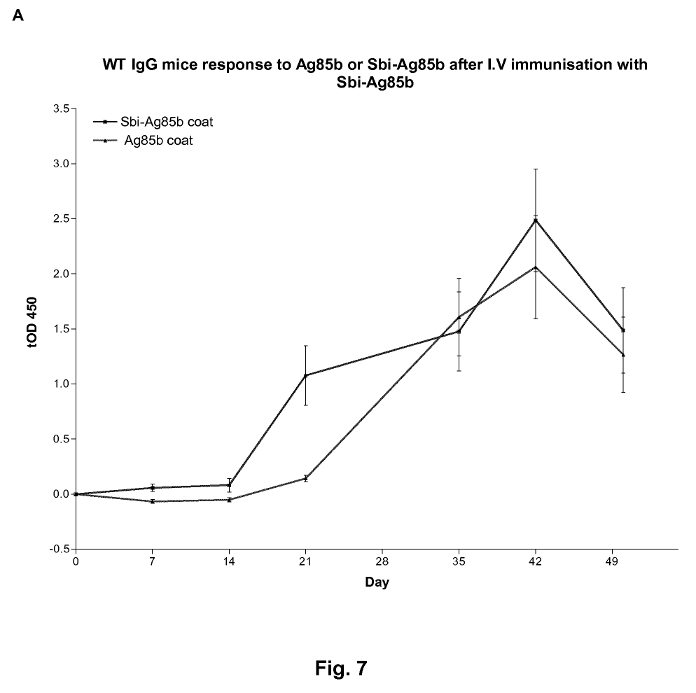

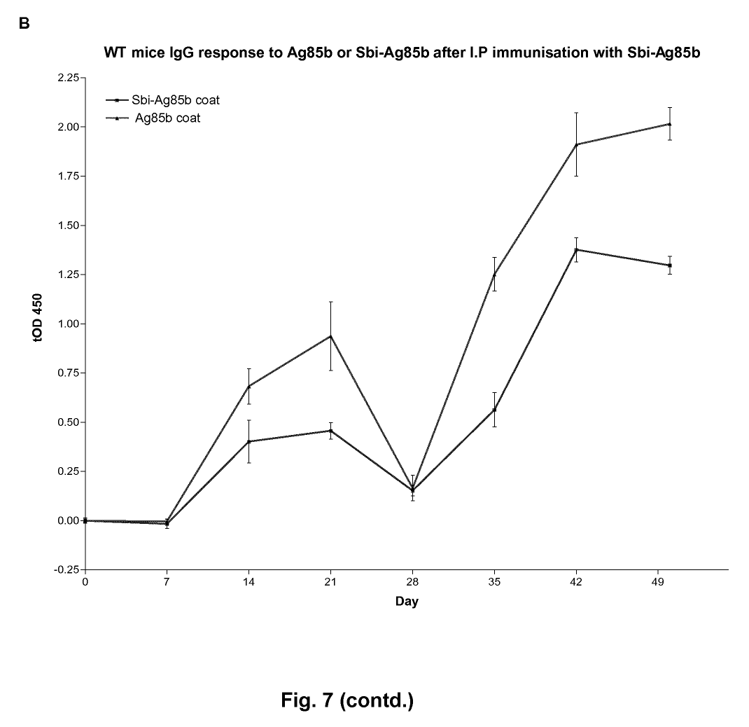

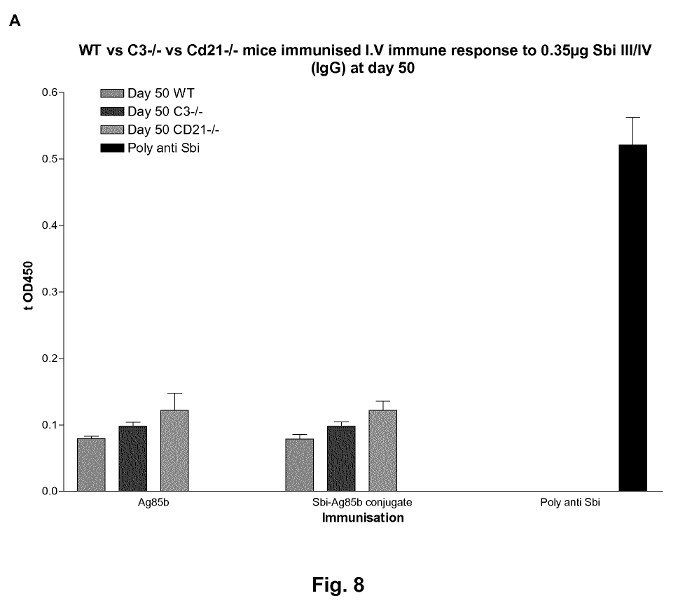

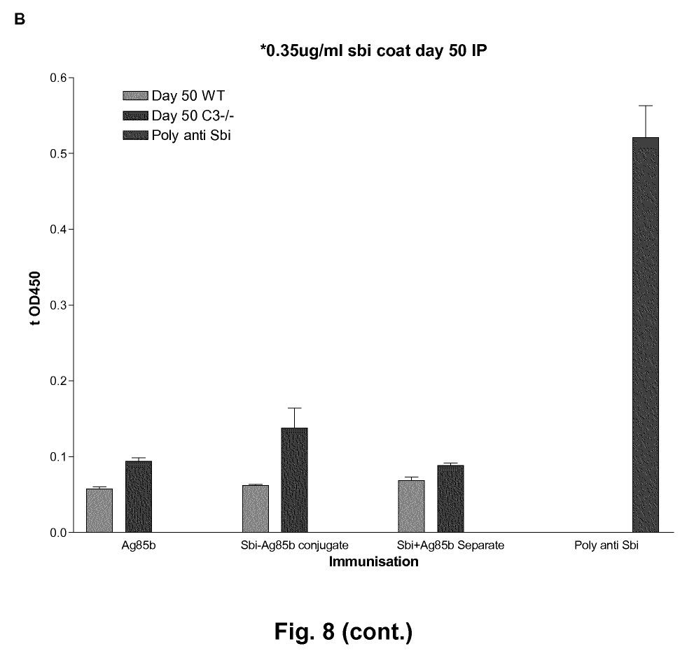

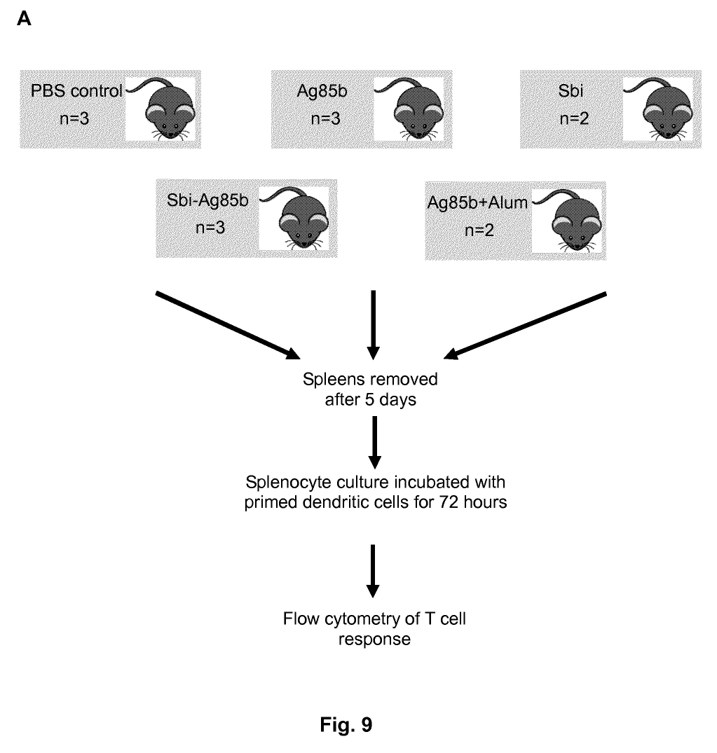

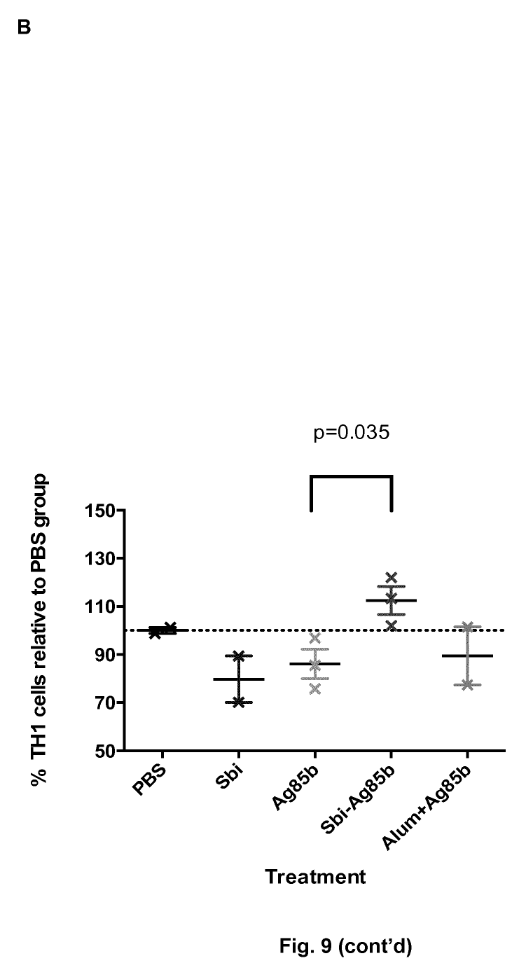

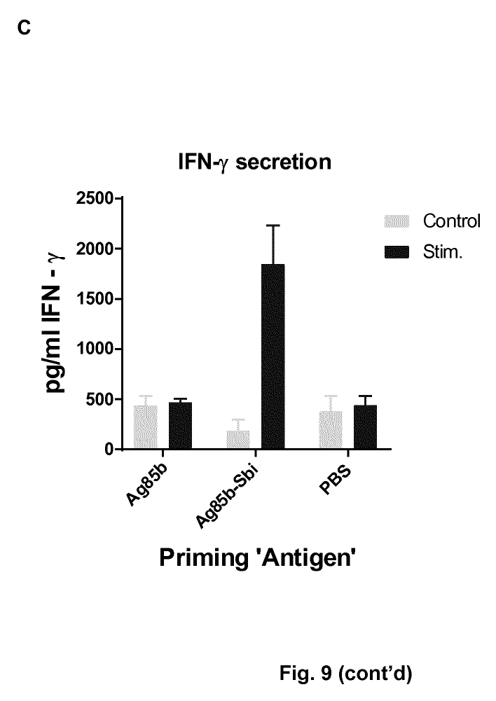

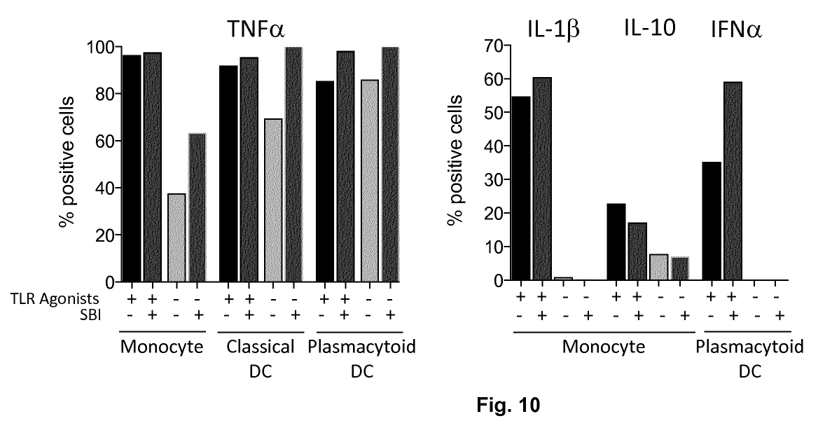

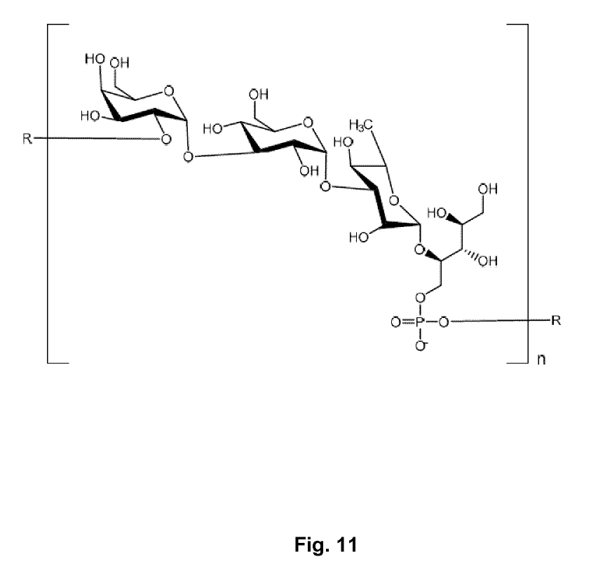

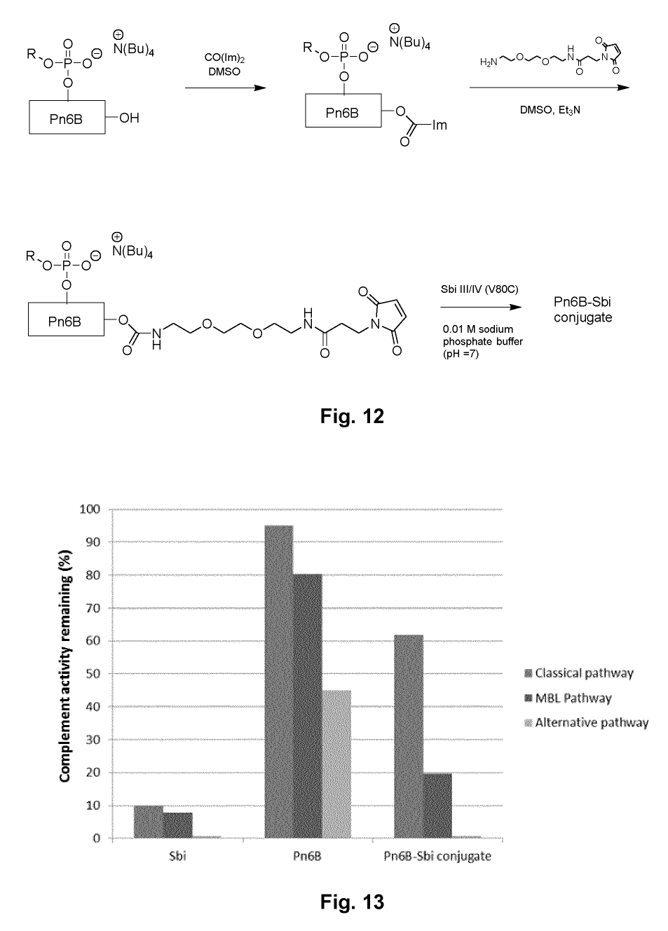

A: Experiment 1 (I.P.1) and experiment 3 (I.P.2). Serum IgG reactivity to Ag85b over time in WT mice when immunised and boosted (day 28) intraperitoneally with either 1.35 μg Sbi-III-IV-Ag85b or 2.7 μg Sbi-III-IV-Ag85b as detected by ELISA.

B: Experiment 1 (I.P.1) and Experiment 2 (I.V). Serum IgG reactivity to Ag85b over time in WT mice when immunised and boosted (day 28) 1.35 μg Sbi-III-IV-Ag85b either intravenously or intraperitoneally as detected by ELISA.

Sera was diluted 1/50 and the mean absorbance ±SEM of each mouse group is shown. All data has been normalised to the day 0 average of all WT mice. (Note, experiment I.P.1 was terminated at day 42 as opposed to day 50).

FIG. 6:

A: Experiment 2 (I.V.). Serum IgG reactivity to Ag85b over time in WT, C3^{-/-}, and CD21^{-/-} mice when immunised and boosted (day 28) intravenously with either 1 μg Ag85b or 1.35 μg Sbi-III-IV-Ag85b as detected by ELISA.

B: Experiment 2 (I.V.). Serum IgG reactivity to Ag85b at day 42 in WT, C3^{-/-}, and CD21^{-/-} mice when immunised and boosted (day 28) intravenously with either 1 μg Ag85b or 1.35 μg Sbi-III-IV-Ag85b as detected by ELISA

C: Experiment 3 (I.P.2). Serum IgG reactivity to Ag85b at day 42 in WT and C3^{-/-} mice when immunised and boosted (day 28) intraperitoneally with either 1 μg Ag85b or 1.35 μg Sbi-III-IV-Ag85b as detected by ELISA. Sera from all IV days 0-28 and all IP was diluted 1/50 and sera from IV days 35-50 was diluted 1/100 and multiplied by 2. The mean absorbance ±SEM of each mouse group is shown. All data has been normalised to the day 0 average of all mice in each group. Note, no blood sample was taken on the boost day 28 due to the IV administration of the vaccine.

FIG. 7:

A: Experiment 2 (I.V.). Serum IgG reactivity to Ag85b or Sbi-III-IV-Ag85b over time in WT mice when immunised and boosted (day 28) intravenously with 1.35 μg Sbi-III-IV-Ag85b as detected by ELISA.

B: Experiment 3 (I.P.2) Serum IgG reactivity to Ag85b or Sbi-III-IV-Ag85b over time in WT mice when immunised and boosted (day 28) intraperitoneally with 2.7 μg Sbi-III-IV-Ag85b as detected by ELISA.

Sera from days 0-28 was diluted 1/50 and sera from days 35-50 was diluted 1/100 and multiplied by 2. The mean absorbance ±SEM of each mouse group is shown. All data has been normalised to the day 0 average of all WT mice

FIG. 8:

A: Experiment 2 (I.V.). Serum IgG reactivity to Sbi-III-IV at day 50 for all mice in IV experiment. Positive control 1/100 polyclonal anti-Sbi serum.

B: Experiment 3 (I.P.2). Serum IgG reactivity to Sbi-III-IV at day 50 for all mice in IP2 experiment. Positive control 1/100 polyclonal anti Sbi serum.

Serum was diluted 1/50 and data has been normalised to day 0 average of all mice in each group. The mean absorbance ±SEM of each mouse group is shown.

FIG. 9:

T cell activation by murine splenocytes primed by antigen administration in vivo.

A: Schematic of experimental design.

B: Activation of Th1 cells.

C: Production of IFN-γ.

FIG. 10:

Intracellular cytokine production in subsets of PBMCs treated in vitro with various combinations of Toll-like receptor ligands (TLRs) and Sbi-III-IV. First panel: TNFα production by monocytes, classical dendritic cells, and plasmacytoid dendritic cells. Second panel: IL-1β and IL-10 production by monocytes and IFNα production by plasmacytoid dendritic cells.

FIG. 11:

Illustration of the (→2-α-D-Galactopyranose-(1→3)-α-D-Glucopyranose-(1→3)-α-L-rhamnopyranose-(1→4)-D-ribitol-5-phosphate-0 repeat unit of Pn6B.

FIG. 12:

Scheme showing the synthetic method employed for the production of Sbi-III-IV(V80C)-Pn6B conjugate.

FIG. 13:

Determination of residual complement (% activity remaining) following complement depletion by incubation with Sbi-III-IV, Pn6B and Sbi-III-IV(V80C)-Pn6B conjugate for the classical (CP), mannose-binding lectin (MBLP), and alternative (AP) complement pathways. Complement depletion (% activity remaining) was quantified from the equation: (sample−negative control)/(positive control−negative control)×100%.

### DETAILED DESCRIPTION OF THE INVENTION

### Complement

The complement system is a crucial part of the innate immune system and consists of a group of approximately 20 proteins, mostly found in the serum. When the system is activated, a cascade of sequential enzyme activation takes place, in which the product of one reaction is itself an enzyme which catalyses the next stage of activation. The cascade thus contains a number of points at which exponential signal amplification occurs, potentially resulting in a massive response from a very small initial stimulus.

The system has three known activation mechanisms, referred to as the classical pathway, the alternative pathway, and the lectin pathway. In simple terms, these three pathways converge into a common downstream effector or “terminal” pathway.

Activation by any one of the three mechanisms has three main effects. Firstly, it results in generation of small protein fragments called anaphylatoxins, which serve as chemoattractants to recruit immune cells to the site of activation. Anaphylatoxins include the components C3a and C5a. Secondly, foreign substances (such as microorganisms, viruses, etc.) which trigger the complement cascade are marked for destruction by coating with so-called opsonins, which become covalently bound to hydroxyl and amine groups on the foreign surface. These opsonins (which include C3b, described in more detail below) are recognised by phagocytic cells such as neutrophils. Thirdly, a complex called the membrane attack complex (MAC), comprising the complement components C5b-C9, may be formed in the membrane of foreign cells leading to membrane lysis.

The protein C3 is a crucial component of all three complement activation pathways. In its intact form it consists of an alpha chain and a beta chain linked by a disulphide bridge. The alpha chain contains an unusual internal thioester bond between Cys1010 and Gln1013 which is exposed by cleavage of C3 during the complement activation process. This thioester bond can then be cleaved by nucleophilic attack from a suitable group (e.g. a hydroxyl or amine group), leading to the formation of a covalent adduct between C3b and the nucleophile, which is an important part of the opsonisation process.

C3b also participates in the formation of an enzyme capable of further C3 cleavage (a “C3 convertase”). iC3b is an inactivated form of C3b formed when C3b is cleaved by a control protein which prevents excessive activation of the complement cascade. C3c, C3dg and C3d are further downstream cleavage products of iC3b. C3d also acts as an opsonin.

Thus, activation of complement at a given site typically results in the generation of complement activation products such as anaphylatoxins and opsonins, which provide signals to various immune cell types here termed “responder” cells. Responder cells are primarily cells of the immune system such as basophils, neutrophils, mast cells and macrophages. Anaphylatoxins and opsonins trigger various functions in these cell types such as chemotaxis (towards the site of complement activation), mast cell degranulation, activation of respiratory burst, phagocytosis of opsonised targets, etc. Opsonisation of a particular target typically results in enhanced generation of antibodies against that target.

Any system comprising C3 always shows a low level of complement activation via spontaneous hydrolysis of C3 (referred to as “tick-over” C3 activation). However the cascade is normally kept in check by powerful regulatory mechanisms.

### Complement-Activating Moiety

The complement-activating moiety comprises an Sbi-III domain and an Sbi-IV domain which together are capable of activating the complement cascade. Without wishing to be bound by theory, it is believed that, when acting in concert, these domains undergo a trans-acylation reaction with C3, thus forming a covalent adduct with C3b. The adduct thus formed is then able to drive localised complement activation (which may involve the formation of a C3 convertase incorporating Factor B, Factor D and further C3b components) leading to opsonisation of the target antigen.

Native Sbi protein is composed (from N- to C-terminus) of a leader peptide, domains Sbi-I, II, III and IV, a putative wall-anchoring sequence (WR) and a so-called Y region. Its sequence, including signal sequence, is as follows:

```
Met Lys Asn Lys Tyr Ile Ser Lys Leu Leu Val Gly
1 5 10
Ala Ala Thr Ile Thr Leu Ala Thr Met Ile Ser Asn
                        15 20
Gly Glu Ala Lys Ala Ser Glu Asn Thr Gln Gln Thr
25 30 35
Ser Thr Lys His Gln Thr Thr Gln Asn Asn Tyr Val
                        40 45
Thr Asp Gln Gln Lys Ala Phe Tyr Gln Val Leu His
        50 55 60
Leu Lys Gly Ile Thr Glu Glu Gln Arg Asn Gln Tyr
                                65 70
Ile Lys Thr Leu Arg Glu His Pro Glu Arg Ala Gln
                75 80
Glu Val Phe Ser Glu Ser Leu Lys Asp Ser Lys Asn
85 90 95
Pro Asp Arg Arg Val Ala Gln Gln Asn Ala Phe Tyr
                        100 105
Asn Val Leu Lys Asn Asp Asn Leu Thr Glu Gln Glu
        110 115 120
Lys Asn Asn Tyr Ile Ala Gln Ile Lys Glu Asn Pro
                                125 130
Asp Arg Ser Gln Gln Val Trp Val Glu Ser Val Gln
                135 140
Ser Ser Lys Ala Lys Glu Arg Gln Asn Ile Glu Asn
145 150 155
Ala Asp Lys Ala Ile Lys Asp Phe Gln Asp Asn Lys
                        160 165
Ala Pro His Asp Lys Ser Ala Ala Tyr Glu Ala Asn
        170 175 180
Ser Lys Leu Pro Lys Asp Leu Arg Asp Lys Asn Asn
                                185 190
Arg Phe Val Glu Lys Val Ser Ile Glu Lys Ala Ile
                195 200
Val Arg His Asp Glu Arg Val Lys Ser Ala Asn Asp
205 210 215
Ala Ile Ser Lys Leu Asn Glu Lys Asp Ser Ile Glu
                        220 225
Asn Arg Arg Leu Ala Gln Arg Glu Val Asn Lys Ala
        230 235 240
Pro Met Asp Val Lys Glu His Leu Gln Lys Gln Leu
                                245 250
Asp Ala Leu Val Ala Gln Lys Asp Ala Glu Lys Lys
                255 260
Val Ala Pro Lys Val Glu Ala Pro Gln Ile Gln Ser
265 270 275
Pro Gln Ile Glu Lys Pro Lys Val Glu Ser Pro Lys
                        280 285
Val Glu Val Pro Gln Ile Gln Ser Pro Lys Val Glu
        290 295 300
Val Pro Gln Ser Lys Leu Leu Gly Tyr Tyr Gln Ser
                                305 310
Leu Lys Asp Ser Phe Asn Tyr Gly Tyr Lys Tyr Leu
                315 320
Thr Asp Thr Tyr Lys Ser Tyr Lys Glu Lys Tyr Asp
325 330 335
Thr Ala Lys Tyr Tyr Tyr Asn Thr Tyr Tyr Lys Tyr
                        340 345
Lys Gly Ala Ile Asp Gln Thr Val Leu Thr Val Leu
        350 355 360
Gly Ser Gly Ser Lys Ser Tyr Ile Gln Pro Leu Lys
                                365 370
Val Asp Asp Lys Asn Gly Tyr Leu Ala Lys Ser Tyr
                375 380
Ala Gln Val Arg Asn Tyr Val Thr Glu Ser Ile Asn
385 390 395
Thr Gly Lys Val Leu Tyr Thr Phe Tyr Gln Asn Pro
                        400 405
Thr Leu Val Lys Thr Ala Ile Lys Ala Gln Glu Thr
        410 415 420
Ala Ser Ser Ile Lys Asn Thr Leu Ser Asn Leu Leu
                                425 430
Ser Phe Trp Lys.
                435
```

By “Sbi-III domain” is meant a polypeptide sequence comprising at least amino acids 150 to 197 of the wild type Sbi sequence, a fragment thereof, or a variant of either having at least 80%, at least 85%, at least 90%, at least 95% or at least 99% sequence identity with the corresponding Sbi sequence. The Sbi-III domain may be at least 30, at least 35, at least 40, or at least 45 amino acids in length. In some embodiments the Sbi-III domain has at least 80%, at least 85%, at least 90%, at least 95% or at least 99% sequence identity with the wild type Sbi-III sequence. In some embodiments, the Sbi-III domain comprises the wild type Sbi-III sequence.

It may be desirable that residue K173 is not modified, as modification at this site may adversely affect Sbi's ability to activate complement.

Wild type Sbi-III (residues 150 to 197 of the molecule shown above) has the sequence:

ERQNIENADKAIKDFQDNKAPHDKSAAYEANSKLPKDLRDKNNRFVEK

By “Sbi-IV domain” is meant a polypeptide sequence comprising at least amino acids 198 to 266 of the Sbi sequence, a fragment thereof, or a variant of either having at least 80%, at least 85%, at least 90%, at least 95% or at least 99% sequence identity with the corresponding Sbi sequence. The Sbi-IV domain retains the ability to bind to C3 protein, especially the C3d portion of C3. Interactions between Sbi-IV and C3 are described by Clark et al., Mol. Immunol. 48(2011), 452-462. The Sbi-IV domain may be at least 30, at least 35, at least 40, at least 45, at least 50, at least 55, at least 60, or at least 65 amino acids in length. In some embodiments the Sbi-IV domain has at least 80%, at least 85%, at least 90%, at least 95% or at least 99% sequence identity with the wild type Sbi-IV sequence. In some embodiments, the Sbi-IV domain comprises the wild type Sbi-IV sequence.

It may be desirable that residues 5199, 5226, R231, N238, K250, K259, K263 and/or K264 are not modified, as modification at these sites may adversely affect Sbi's ability to form a covalent adduct with C3b and/or otherwise to activate complement.

Wild type Sbi-IV (residues 198 to 266 of the molecule shown above) has the sequence:

VSIEKAIVRHDERVKSANDAISKLNEKDSIENRRLAQREVNKAPMDVKEH
LQKQLDALVAQKDAEKKVA

The Sbi-III and Sbi-IV sequences may be contiguous as in the native protein. For example, residues 150-266 of the Sbi molecule shown above (i.e. wild type Sbi-III-IV) have the sequence:

ERQNIENADKAIKDFQDNKAPHDKSAAYEANSKLPKDLRDKNNRFVEKVS
IEKAIVRHDERVKSANDAISKLNEKDSIENRRLAQREVNKAPMDVKEHLQ
KQLDALVAQKDAEKKVA

The Sbi-III-IV moiety may therefore comprise or consist of residues 150-266 of the Sbi sequence shown, or a fragment thereof capable of undergoing the required transacylation reaction with C3. Alternatively the complement-activating moiety may comprise a variant of either which has at least 80%, at least 85%, at least 90%, at least 95% or at least 99% sequence identity with the corresponding Sbi sequence and which retains the ability to undergo the required transacylation reaction with C3. In some embodiments the Sbi-III-IV moiety has at least 80%, at least 85%, at least 90%, at least 95% or at least 99% sequence identity with the wild type Sbi-III-IV sequence.

An example of a modification to the wild type Sbi-III-IV sequence is the introduction of a cysteine residue, e.g. to facilitate covalent linkage to a target antigen, especially a non-peptide target antigen such as a carbohydrate, polysaccharide, lipopolysaccharide or lipid. For example, the valine residue adjacent the C-terminus of Sbi-III-IV protein (V68 of Sbi-IV; V116 of Sbi-III-IV) may be mutated to cysteine to yield the construct was designated Sbi-III-IV(V80C) in the Examples below, which has the sequence:

ERQNIENADKAIKDFQDNKAPHDKSAAYEANSKLPKDLRDKNNRFVEKVS
IEKAIVRHDERVKSANDAISKLNEKDSIENRRLAQREVNKAPMDVKEHLQ
KQLDALVAQKDAEKKCA

In some embodiments, the Sbi-III-IV moiety comprises the wild type Sbi-III-IV sequence.

Alternatively, the Sbi-III and Sbi-IV domains may be separated by a linker sequence. As described elsewhere, peptide linkers are typically between 12 and 30 amino acids in length, and have a high proportion of small and hydrophilic amino acid residues (e.g. glycine and serine) to provide the required flexibility without compromising solubility, and may comprise additional elements such as poly-His sequences (e.g. His₆-His₁₀).

In some embodiments, the complement-activating moiety is not capable of binding to an immunoglobulin Fc region, i.e. it has substantially no affinity for an immunoglobulin Fc region. For example, it does not comprise a domain having affinity for Fc.

In some embodiments, the complement-activating moiety does not comprise any further Sbi sequence other than an Sbi-III domain and an Sbi-IV domain. For example, it does not comprise either an Sbi-I domain or an Sbi-II domain. However, if desired, it may comprise an Sbi-I domain or an Sbi-II domain.

By “Sbi-I domain” is meant a polypeptide sequence comprising amino acids 42 to 90 of the Sbi sequence shown above, a fragment thereof, or a variant of either having at least 80% sequence identity with the corresponding Sbi sequence. The Sbi-I domain may retain the ability to bind immunoglobulin, in particular via the Fc region.

By “Sbi-II domain” is meant a polypeptide sequence comprising at least amino acids 92 to 149 of the Sbi sequence shown above, a fragment thereof, or a variant of either having at least 80% sequence identity with the corresponding Sbi sequence. The Sbi-II domain may retain the ability to bind immunoglobulin, in particular via the Fc region.

Percent (%) amino acid sequence identity with respect to a reference sequence is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. % identity values may be determined by WU-BLAST-2 (Altschul et al., Methods in Enzymology, 266:460-480 (1996)). WU-BLAST-2 uses several search parameters, most of which are set to the default values. The adjustable parameters are set with the following values: overlap span=1, overlap fraction=0.125, word threshold (T)=11. A % amino acid sequence identity value is determined by the number of matching identical residues as determined by WU-BLAST-2, divided by the total number of residues of the reference sequence (gaps introduced by WU-BLAST-2 into the reference sequence to maximize the alignment score being ignored), multiplied by 100.

Reference to “corresponding Sbi sequence” should be taken to mean the portion of Sbi sequence which aligns with a query sequence when that query sequence is optimally aligned with the full length Sbi sequence. Thus, for example, a 40 amino acid sequence which is identical to a contiguous 40 amino acid stretch of Sbi-III would be considered to have 100% identity to that stretch of corresponding Sbi sequence.

It may be desirable (although not necessary) that the Sbi domains used for the purposes of the present differ from the Sbi sequence shown only by conservative substitutions.

Conservative substitutions may be defined as substitutions within the following groups of amino acids:

I. Asp and Glu (acidic amino acids);
II. Arg, Lys and His (basic amino acids);
III. Asn, Gln, Ser, Thr and Tyr (uncharged polar amino acids);
IV. Ala, Gly, Val, Leu, Ile, Pro, Phe, Met, Trp and Cys (non-polar amino acids).

The complement-activating moieties described here may bind C3 (and form adducts with C3b) from any mammalian species, including rodents (e.g. mice, rats), lagomorphs (e.g. rabbits), felines (e.g. cats), canines (e.g. dogs), equines (e.g. horses), bovines (e.g. cows), caprines (e.g. goats), ovines (e.g. sheep), other domestic, livestock or laboratory animals, or primates (e.g. Old World monkey, New World monkey, apes or humans). Preferably they bind human C3 protein.

As discussed elsewhere in this specification, the complement-activating moiety is capable of enhancing an immune response against a target antigen, as compared to an equivalent administration in the absence of complement-activating moiety, or an equivalent administration with an inactivate analogue of the complement-activating moiety, i.e. one which is not capable of activating complement. Such analogues may have an identical amino acid sequence to the complement-activating moiety but have been physically inactivated, e.g. by incorrect folding, heat treatment, or other modes of denaturation. Alternatively, the analogue may differ from the complement activating-moiety by one or more point mutations in the Sbi-III-IV sequence, e.g. at one or more of positions K173, 5199, R213, N238. The substituent amino acid may be Ala or any other residue which results in an inactive analogue, i.e. suitable substitutions include K173A, S199A, R213A and N238A. Thus, a reference inactivate analogue of any given complement-activating moiety may be generated by introducing one of these modifications, or more if desired. Preferably, the analogue differs from the complement-activating moiety in question only by one such modification.

### Target Antigen

The target antigen may be any antigen against which it is desirable to raise an immune response, and especially any antigen against which it is desirable to stimulate production of antibodies, particularly IgG.

The target antigen may be a peptide antigen. As mentioned above, the term “peptide” here refers to the nature of the antigen, i.e. that it is formed from amino acids linked by peptide bonds, and should not be taken to imply any particular size or length. Typically the peptide antigen will be at least 8 amino acids in length, and may be up to 30 amino acids in length, up to 50 amino acids in length, up to 100 amino acids, up to 200 amino acids, or even longer. It may be a complete protein, an isolated domain of a protein, or a peptide fragment of a protein.

Alternatively, the antigen may be a non-peptide antigen. It may comprise or consist of any other type of biological molecule, including a carbohydrate, polysaccharide, lipid, lipopolysaccharide, etc. For example a non-peptide antigen may be a polysaccharide or lipopolysaccharide from the cell membrane, cell wall or capsule of an infectious organism, such as the polysaccharide Pn6B from Streptococcus pneumoniae.

The target antigen may be covalently linked (e.g. chemically conjugated) to the complement-activating moiety. Chemical conjugation may be performed by any suitable means and the skilled person will be well aware of suitable technologies, including, but not limited to: (1) direct coupling via protein functional groups (e.g., thiol-thiol linkage, amine-carboxyl linkage, amine-aldehyde linkage; enzyme direct coupling); (2) homobifunctional coupling of amines (e.g., using bis-aldehydes); (3) homobifunctional coupling of thiols (e.g., using bis-maleimides); (4) homobifunctional coupling via photoactivated reagents (5) heterobifunctional coupling of amines to thiols (e.g., using maleimides); (6) heterobifunctional coupling via photoactivated reagents (e.g., the β-carbonyldiazo family); (7) introducing amine-reactive groups into a poly- or oligosaccharide via cyanogen bromide activation or carboxymethylation; (8) introducing thiol-reactive groups into a poly- or oligosaccharide via a heterobifunctional compound such as maleimido-hydrazide; (9) protein-lipid conjugation via introducing a hydrophobic group into the protein and (10) protein-lipid conjugation via incorporating a reactive group into the lipid. Also, contemplated are heterobifunctional “non-covalent coupling” techniques such the biotin-avidin interaction. For a comprehensive review of conjugation techniques, see Aslam and Dent (1998). Thus the antigen may be covalently linked to the side chain of a residue of the Sbi-III-IV moiety, e.g. to the side chain of a cysteine residue.

Where the target antigen is a peptide antigen, it may be provided as part of a fusion protein with the complement-activating moiety; i.e. the antigen and complement-activating moiety are part of the same peptide chain.

In the fusion protein, the target antigen may be N-terminal of the complement-activating moiety, or the complement-activating moiety may be N-terminal of the target antigen.

The fusion protein may comprise other components. For example, it may comprise a linker peptide between the complement-activating moiety and the target antigen.

A peptide linker is typically between 12 and 30 amino acids in length, with a high proportion of small and hydrophilic amino acid residues (e.g. glycine and serine) to provide the required flexibility without compromising aqueous solubility of the molecule. For example, it may comprise at least 50% glycine and serine residues, at least 60% glycine and serine residues, at least 70% glycine and serine residues, at least 80% glycine and serine residues, or at least 90% glycine and serine residues.

Additionally or alternatively, the fusion protein may comprise a peptide tag (e.g. a poly-His sequence, such as His₆-His₁₀) to facilitate purification. Such a tag may, for example, be located at the N-terminus or the C-terminus of the fusion protein, or within a linker sequence. By way of illustration, in the examples below, a linker is employed between a complement-activating moiety (Sbi-III-IV) and a target antigen (Ag85b) which contains a poly-His tag, and has the sequence GTSGGGGSHHHHHHHHHHSGGGGS.

The target antigen itself will be chosen depending on the nature of the desired immune response.

It may be desirable to generate an immune response against an infectious organism, e.g. for prophylaxis or treatment of infection by that organism. Thus the target antigen may be derived from an infectious organism, such as a bacterium, fungal cell, virus, protozoan, or other parasite. In this context, “derived from” means genetically encoded, expressed, or otherwise synthesised by the infectious organism. Typically, the target antigen will be expressed or otherwise displayed on the surface of that organism.

A bacterial target antigen may be from a gram positive bacterium or a gram negative bacterium.

A bacterial target antigen may be from a bacterium of one of the following genera or species, which include common human pathogens:

Actinomyces (e.g. Actinomyces israelii);
Bacillus (e.g. Bacillus anthracis, Bacillus cereus);
Bacteroides (e.g. Bacteroides fragilis);
Bartonella (e.g. Bartonella henselae, Bartonella quintana);
Bordetella (e.g. Bordetella pertussis);
Borrelia (e.g. Borrelia burgdorferi, Borrelia garinii, Borrelia afzelii, Borrelia recurrentis);
Brucella (e.g. Brucella abortus, Brucella canis, Brucella melitensis, Brucella suis);
Campylobacter (e.g. Campylobacter jejuni);
Chlamydia and Chlamydophila (e.g. Chlamydia pneumoniae Chlamydia trachomatis Chlamydophila psittaci);
Clostridium (e.g. Clostridium botulinum, Clostridium difficile, Clostridium perfringens Clostridium tetani);
Corynebacterium (e.g. Corynebacterium diphtheriae);
Cryptococcus (e.g. Cryptococcus neoformans);
Ehrlichia (e.g. Ehrlichia canis, Ehrlichia chaffensis);
Enterococcus (e.g. Enterococcus faecalis, Enterococcus faecium);
Escherichia (e.g. Escherichia coli);
Francisella (e.g. Francisella tularensis);
Haemophilus (e.g. Haemophilus influenzae);
Helicobacter (e.g. Helicobacter pylori);
Klebsiella (e.g. Klebsiella pneumoniae);
Legionella (e.g. Legionella pneumophila);
Leptospira (e.g. Leptospira interrogans, Leptospira santarosai, Leptospira weilii, Leptospira noguchii);
Listeria (e.g. Listeria monocytogenes);
Mycobacterium (e.g. Mycobacterium leprae, Mycobacterium tuberculosis, Mycobacterium ulcerans);
Mycoplasma (e.g. Mycoplasma pneumoniae);
Neisseria (e.g. Neisseria gonorrhoeae, Neisseria meningitidis);
Nocardia (e.g. Nocardia asteroides);
Pseudomonas (e.g. Pseudomonas aeruginosa);
Rickettsia (e.g. Rickettsia rickettsii);
Salmonella (e.g. Salmonella typhi, Salmonella typhimurium, Salmonella enterica);
Shigella (e.g. Shigella sonnei, Shigella dysenteriae, Shigella flexneri);
Staphylococcus (e.g. Staphylococcus aureus, Staphylococcus epidermidis, Staphylococcus saprophyticus);
Streptococcus (e.g. Streptococcus agalactiae, Streptococcus pneumoniae, Streptococcus pyogenes, Streptococcus viridans);
Treponema (e.g. Treponema pallidum);
Ureaplasma (e.g. Ureaplasma urealyticum);
Vibrio (e.g. Vibrio cholerae);
Yersinia (e.g. Yersinia pestis, Yersinia enterocolitica, Yersinia pseudotuberculosis).

In some embodiments, the antigen is not derived from a Staphylococcus species (i.e. it is not a Staphylococcal antigen). In some embodiments, the antigen is not derived from Staphylococcus aureus or from Staphylococcus epidermidis.

A fungal target antigen may be from one of the following genera or species, which include common human pathogens:

Candida (e.g. Candida albicans, Candida glabrata, Candida rugosa, Candida parapsilosis, Candida tropicalis, Candida dubliniensis);
Aspergillus (e.g. Aspergillus fumigatus, Aspergillus flavus);
Cryptococcus (e.g. Cryptococcus neoformans);
Histoplasma (e.g. Histoplasma capsulatum);
Pneumocystis (e.g. Pneumocystis jirovecii, Pneumocystis carinii);
Stachybotrys (e.g. Stachybotrys charatum).

A viral target antigen may be derived from one of the following virus families or species, which include common human pathogens:

Adenoviridae (e.g. Adenovirus);
Herpesviridae (e.g. Herpes simplex, type 1, Herpes simplex, type 2, Varicella-zoster virus, Epstein-Barr virus, Human cytomegalovirus, Human herpesvirus type 8);
Papillomaviridae (e.g. Human papillomavirus);
Polyomaviridae (e.g. BK virus, JC virus);
Poxviridae (e.g. Smallpox);
Hepadnaviridae (e.g. Hepatitis B virus);
Parvoviridae (e.g. Parvovirus B19);
Astroviridae (e.g. Human astrovirus);
Caliciviridae (e.g. Norwalk virus);
Picornaviridae (e.g. coxsackievirus, hepatitis A virus, poliovirus, rhinovirus);
Coronaviridae (e.g. Severe acute respiratory syndrome virus);
Flaviviridae (e.g. Hepatitis C virus, yellow fever virus, dengue virus, West Nile virus, TBE virus);
Togaviridae (e.g. Rubella virus);
Hepeviridae (e.g. Hepatitis E virus);
Retroviridae (e.g. Human immunodeficiency virus (HIV), Human T-cell leukaemia virus (HTLV) types I, II, III and IV);
Orthomyxoviridae (e.g. Influenza virus);
Arenaviridae (e.g. Lassa virus);
Bunyaviridae (e.g. Crimean-Congo hemorrhagic fever virus, Hantaan virus);
Filoviridae (e.g. Ebola virus, Marburg virus);
Paramyxoviridae (e.g. Measles virus, Mumps virus, Parainfluenza virus, Respiratory syncytial virus); Rhabdoviridae (e.g. Rabies virus);
Hepatitis D virus;
Reoviridae (e.g. Rotavirus, Orbivirus, Coltivirus, Banna virus).

A protozoan target antigen may be derived from one of the following genera or species of protozoan, which include common human pathogens:

Plasmodium spp. (responsible for malaria, e.g. Plasmodium falciparum, Plasmodium berghei, Plasmodium yoelii, Plasmodium vivax and Plasmodium knowlesii)
Entamoeba (Entamoeba histolytica is responsible for amoebic dysentery);
Giardia (responsible for Giardiasis, e.g. Giardia lamblia);
trypanosomes (e.g. Trypanosoma brucei, which causes African sleeping sickness, and Trypanosoma cruzi);
Leishmania (e.g. Leishmania spp. and Leishmania mexicana);
Toxoplasma (e.g. Toxoplasma gondii);

### Acanthamoeba;

### Babesia;

Balamuthia (e.g. Balamuthia mandrillaris)

### Cryptosporidium;

### Cyclospora;

Naegleria (e.g. Naegleria fowleri).

Some of these organisms (e.g. Plasmodium, trypanosomes, Leishmania and Toxoplasma gondii) may also infect host cells.

Other parasites from which the target antigen may be derived include helminths (e.g. Ascaris lumbricoides, pinworm, Strongyloides stercoralis, Toxocara, guinea worm, hookworm, tapeworm, whipworm) and flukes (e.g. Schistosoma, Gnathostoma, Paragonimus, Fasciola hepatica, Trichobilharzia regenti).

It may also be beneficial to stimulate an immune response against a neoplastic cell. The neoplastic cell may be benign or malignant. It may be a cancer cell.

Thus, the target antigen may be a marker expressed specifically or preferentially on a neoplastic cell, e.g. a cancer cell. Such a marker may be referred to as a “tumour-specific marker” or “tumour-specific antigen”, although their expression is not restricted to solid tumours.

Markers expressed on neoplastic cells may be “self” antigens. Thus the target antigen may be derived from the same species as the subject to which it is to be administered, or derived from the subject themselves.

Examples of “self” cancer antigens include alphafetoprotein (AFP, found in germ cell tumours and hepatocellular carcinoma), carcinoembryonic antigen (CEA, found in bowel cancers and certain lung and breast cancers), CA-125 (found in ovarian cancer), MUC-1 (found in breast cancer), epithelial tumour antigen (ETA, found in breast cancer), tyrosinase (found in malignant melanoma), melanoma-associated antigen (MAGE, found in malignant melanoma) and variants of Ras and p53.

Markers of neoplasia may alternatively be “non-self”, e.g. derived from an infectious organism associated with (or causative of) the neoplasia. Many neoplasias and cancers are associated with or caused by oncoviruses. Such conditions and their associated viruses include hepatocellular carcinoma (hepatitis viruses including hepatitis B and C), tropical spastic paraparesis and adult T cell leukaemia (human T-lymphotropic virus [HTLV]), cancers of the cervix, anus, penis, vulva/vagina and oropharyngeal cancer (human papillomaviruses), Kaposi's sarcoma, multicentric Castleman's disease and primary effusion lymphoma (Kaposi's sarcoma-associated herpesvirus [HHV-8]), Merkel cell carcinoma (Merkel cell polyomavirus), and Burkitt's lymphoma, Hodgkin's lymphoma, post-transplantation lymphoproliferative disease and nasopharyngeal carcinoma (Epstein Barr virus [EBV]). Thus the target antigen may be an antigen derived from one of these viruses.

For the avoidance of any doubt, the target antigen is not Sbi. For example, the antigen typically has less than 60% sequence identity with Sbi protein. (That is to say, when optimally aligned with the Sbi sequence set out above, the antigen has less than 60% sequence identity with the corresponding Sbi sequence in the region of overlap.) Preferably, the antigen has less than 50%, less than 40%, less than 30%, less than 25% or less than 20% sequence identity with the corresponding Sbi sequence.

The complement-activating moiety comprises Sbi-III-IV. As discussed above, it may also comprise additional Sbi sequences, including an Sbi-I domain and/or an Sbi-II domain. These sequences are not to be construed to constitute the target antigen.

### Pharmaceutical Compositions and Methods of Treatment

The molecules described herein can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, which may be by any suitable route, and may be oral or parenteral. Because of the difficulties experienced with oral delivery of peptide agents, parenteral administration may prove the most suitable. Suitable parenteral routes include but are not limited to intravenous, intramuscular, intraperitoneal, cutaneous, subcutaneous, transdermal, and other mucosal routes such as nasal, buccal, rectal and vaginal routes. Examples of suitable compositions and methods of administration are provided in Esseku and Adeyeye (2011) and Van den Mooter G. (2006).

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whatever the nature of the active agent that is to be given to an individual (e.g. a cell, polypeptide, nucleic acid molecule, other pharmaceutically useful agent according to the present invention), administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

### EXAMPLES

### Stimulation of Immune Response Against Peptide Antigen

Sbi-III-IV and the Ag85b protein from Mycobacterium tuberculosis were expressed alone, and as a fusion protein.

To facilitate covalent attachment to Pn6B antigen, the valine residue adjacent the C-terminus of Sbi-III-IV protein (V68 of Sbi-IV; V116 of Sbi-III-IV) was mutated to cysteine. The resulting construct was designated Sbi-III-IV(V80C).

All proteins were expressed with poly-His tags to assist purification. Sbi-III-IV, Ag85b, and the Sbi-III-IV-Ag85b fusion protein were expressed in both E. coli and CHO expression systems. Sbi-III-IV(V80C) was expressed only in E. coli. Consistent results were obtained whichever expression system was used.

### Test Proteins

Sbi-III-IV
MRGSHHHHHHGSERQNIENADKAIKDFQDNKAPHDKSAAYEANSKLPKDL
RDKNNREVEKVSIEKAIVRHDERVKSANDAISKLNEKDSIENRRLAQREV
NKAPMDVKEHLQKQLDALVAQKDAEKKVA [Sbi sequence shown
in italics.]
Ag85b
MPLLLLLPLLWAGALAMDVDKLHHHHHHHHHTSASFSRPGLPVEYLQVPS
PSMGRDIKVQFQSGGNNSPAVYLLDGLRACIDDYNGWDINTPAFEWYYQS
GLSIVMPVGGQSSFYSDWYSPACGKAGCQTYKWETFLTSELPQWLSANRA
VKPTGSAAIGLSMAGSSAMILAAYHPQQFIYAGSLSALLDRSQGMGPSLI
GLAMGDAGGYKAADMWGPSSDPAWERNDPTQQIPKLVANNTRLWVYCGNG
TPNELGGANIPAEFLENFVRSSNLKFQDAYNAAGGHNAVFNFPPNGTHSW
EYWGAQLNAMKGDLQSSLGAGKP [Full-length precursor
sequence. Signal peptide underlined. Ag85b
sequence shown in italics.]
Sbi-III-IV-Ag85b fusion
MPLLLLLPLLWAGALAMDVDERQNIENADKAIKDFQDNKAPHDKSAAYEA
NSKLPKDLRDKNNRFVEKVSIEKAIVRHDERVKSANDAISKLNEKDSIEN
RRLAQREVNKAPMDVKEHLQKQLDALVAQKDAEKKVAGTSGGGGSHHHHH
HHHHHSGGGGSTSASFSRPGLPVEYLQVPSPSMGRDIKVQFQSGGNNSPA
VYLLDGLRACIDDYNGWDINTPAFEWYYQSGLSIVMPVGGQSSFYSDWYS
PACGKAGCQTYKWETFLTSELPQWLSANRAVKPTGSAAIGLSMAGSSAMI
LAAYHPQQFIYAGSLSALLDRSQGMGPSLIGLAMGDAGGYKAADMWGPSS
DPAWERNDPTQQIPKLVANNTRLWVYCGNGTPNELGGANIPAEFLENFVR
SSNLKFQDAYNAAGGHNAVFNFPPNGTHSWEYWGAQLNAMKGDLQSSLGA
GKP [Full-length precursor sequence. Signal
peptide underlined. Sbi and Ag85b sequences shown
in italics.]
Sbi-III-IV (V80C)
MRGSHHHHHHGSERQNIENADKAIKDFQDNKAPHDKSAAYEANSKLPKDL
RDKNNRFVEKVSIEKAIVRHDERVKSANDAISKLNEKDSIENRRLAQREV
NKAPMDVKEHLQKQLDALVAQKDAEKK [symbol] A [Sbi-III-IV(V80C)
sequence shown in italics. V to C substitution
shown in bold and underlined.]

### Recombinant Protein Expression

For expression in CHO cells, plasmids encoding the test proteins were transfected into CHO cells using jetPRIME® transfection reagent (Polyplus Transfections). Cells were kept at 37° C. in RPMI 1640 (Lonza) media. One day post-transfection, cells were selected by the addition of 0.8/ml hygromycin. After approximately 1 week, cells were grown in a maintenance level of 0.2 mg/ml hygromycin media. Supernatant was collected when cells were split and kept frozen at −20° C.

Expression in E. coli was performed as described in Burman et al., 2008, J Biol Chem 283, 17579-17593.

### Confirmation of Protein Production

Supernatant from transfected CHO cell cultures was collected and mixed with nickel-agarose beads (Thermo). This was left overnight at 4° C. with constant shaking. Beads were washed twice with nickel-His binding buffer (0.5M NaCl/0.1M Tris-HCl containing 40-80 mM imidazole, pH 8.0) Buffer was removed and beads were mixed 1:1 with non-reducing PAGE buffer and boiled for 5 minutes. Samples were loaded onto a 12.5% SDS-PAGE gel, transferred to nitrocellulose and developed with anti-His and HRPO. If His-tag was shown to be present at approximately 48.5 kDa (size of Sbi-III-IV-Ag85b), cells were serially diluted in a 96 well plate to allow single colonies to grow. Single colonies were then tested using immunoprecipitation as above and positive colonies allowed to grow in larger T175 flasks.

The identity of the bacterially-expressed Sbi_III-IV protein was confirmed by mass spectrometry and Western blotting using polyclonal anti-Sbi antibodies.

### ÄKTA Protein Purification of Supernatant

Supernatant from transfected CHO cells was collected regularly as flasks were split. When sufficient supernatant was collected, it was mixed 1:1 with equilibration buffer (pH 7.4, 50 mM NaH₂PO₄, 300 mM NaCl, 25 mM imidazole), filter sterilised and run through a cobalt column (GE) at a rate of 1 ml/minute at 4° C. The column was then washed with wash buffer (pH 7.4, 50 mM NaH₂PO₄, 300 mM NaCl, 25 mM Imidazole), and eluted with elution buffer (pH 7.4, 50 mM NaH₂PO₄, 300 mM NaCl, 300 mM Imidazole) on an ÄKTA (GE) protein purification system. Fractions of eluted protein at a gradient of up to 100% elution buffer were collected and analysed on SDS-PAGE stained with Coomassie stain, or transferred for western blot developed with mouse anti-poly histidine antibody to confirm the presence of the purified His-tagged protein.

A similar method was used for purification of bacterially-expressed protein, as described in Burman et al., 2008, J Biol Chem 283, 17579-17593.

### Buffer Exchange and Concentration of Purified Protein

Appropriate fractions containing the purified His-tagged protein were combined and concentrated using a 30,000 kDa cut off spin column (Vivaspin) to a final volume of 2.5 ml. The concentrated protein was then buffer exchanged into phosphate buffered saline (PBS) using PD-10 columns (GE). Final yields were measured by nanodrop.

### In Vitro Complement Activation by Sbi-III-IV-Ag85b

A Western blot assay was carried out to confirm that the purified Sbi-III-IV-Ag85b protein was able to activate the complement system in vitro. Fresh mouse serum (CD21^{-/-}) was added to Sbi-III-IV or Sbi-III-IV-Ag85b, ensuring that the amount of Sbi-III-IV in each preparation was equivalent. The reaction was stopped at 0, 30, 60 and 120 minutes, by the addition of reducing sample buffer, boiled for 5 minutes and run on a 10% SDS-PAGE gel. These were transferred to nitrocellulose membranes and developed with rabbit anti C3 ( 1/1000) and goat anti rabbit HRPO ( 1/2000).

### Ensuring Stoichiometry of Proteins in Vaccine Preparations

To ensure administration of equivalent quantities of antigen proteins, mice immunised with Sbi-III-IV-Ag85b received 1.35 μg of protein and those immunised with Ag85b received 1 μg. (Molecular weight of Sbi-III-IV is 17 kDa and that of Ag85b is 3.15 kDa.)

### Experiment 1 (I.P.1)

To determine whether mice immunised with Sbi-III-IV-Ag85b had a higher immune response to Ag85b than those immunised with Ag85b alone, mice were immunised by intraperitoneal (I.P.) injection. Animals receiving antigen Ag85b alone were injected with 1 μg Ag85b in 200 μl 150 mM NaCl and animals receiving Sbi-III-IV-Ag85b were injected with 1.35 μg Sbi-III-IV-Ag85b in 200 μl 150 mM NaCl. Negative controls were mice immunised with 200 μl 150 mM NaCl. Positive control was a WT mouse immunised with 1 μg Ag85b plus Complete Freund's Adjuvant (CFA). C3^{-/-} mice were used as an internal negative control.

Mice were immunised once at day 0 and boosted at day 28. Serum samples (from approximately 70 μl blood) were collected weekly for ELISA analysis until sacrifice by cardiac puncture at day 42.

TABLE 1
Mice used in experiment 1 (I.P.1)

| Genotype | Immunisation | n= |
|---|---|---|
| WT (C57BL/6) | 200 μl 150 mM NaCl (Negative control) | 1 |
| WT (C57BL/6) | 100 μl 0.1 mg/ml Ag85b plus 100 μlCFA | 1 |
| | (Positive control) | |
| WT (C57BL/6) | 1 μg Ag85b in 200 μl 150 mM NaCl | 4 |
| WT (C57BL/6) | 1.35 μgSbi-III-IV-Ag85b in 200 μl | 4 |
| | 150 mM NaCl | |
| C3^{-/-} (C57BL/6) | 200 μl 150 mM NaCl (Negative control) | 2 |
| C3^{-/-} (C57BL/6) | 1 μg Ag85b in 200 μl 150 mM NaCl | 3 |
| C3^{-/-} (C57BL/6) | 1.35 μgSbi-III-IV-Acf85b in 200 μl | 3 |
| | 150 mM NaCl | |

### Experiment 2 (I.V.)

To determine if an alternative route of administration would improve the response to Ag85b, mice were immunised by intravenous (I.V.) injection. Animals receiving antigen Ag85b alone were injected with 1 μg Ag85b in 50 μl 150 mM NaCl and animals receiving Sbi-III-IV-Ag85b were injected with 1.35 μg Sbi-III-IV-Ag85b in 50 μl 150 mM NaCl. C3^{-/-} and CD21^{-/-} mice were used as internal negative controls.

Mice were immunised once at day 0 and boosted at day 28. Serum samples (from approximately 70 μl blood) were collected weekly for ELISA analysis until sacrifice by cardiac puncture at day 50. No sample was taken at the boost day 28 due to the I.V. route of the immunisation.

TABLE 2
Mice used in experiment 2 (I.V.)

| Genotype | Immunisation | n= |
|---|---|---|
| WT (C57BL/6) | 1 μg Ag85b in 200 μl 150 mM NaCl | 5 |
| WT (C57BL/6) | 1.35 μgSbi-III-IV-Ag85b in 200 μl | 5 |
| | 150 mM NaCl | |
| C3^{-/-} (C57BL/6) | 1 μg Ag85b in 200 μl 150 mM NaCl | 4 |
| C3^{-/-} (C57BL/6) | 1.35 μgSbi-III-IV-Ag85b in 200 μl | 4 |
| | 150 mM NaCl | |
| CD21^{-/-} (C57BL/6) | 1 μg Ag85b in 200 μl 150 mM NaCl | 5 |
| CD21^{-/-} (C57BL/6 | 1.35 μgSbi-III-IV-Ag85b in 200 μl | 5 |
| | 150 mM NaCl | |

### Experiment 3 (I.P.2)

To determine if doubling the dose of Sbi-III-IV-Ag85b would improve immune response to Ag85b, mice were immunised by intraperitoneal (I.P.) injection. In this experiment, Ag85b and Sbi-III-IV were also administered together as separate proteins.

Animals receiving antigen Ag85b alone were injected with 2 μg Ag85b in 200 μl 150 mM NaCl and animals receiving Sbi-III-IV-Ag85b protein were injected with 2.7 μg Sbi-III-IV-Ag85b in 200 μl 150 mM NaCl. Mice immunised with Sbi-III-IV and Ag85b as separate proteins were injected with 200 μl 150 mM NaCl containing both 0.7 μg Sbi-III-IV and 2 μg Ag85b, to allow for equivalent amounts of Ag85b. C3^{-/-} mice were used as an internal negative control.

Mice were immunised once at day 0 and boosted at day 28. Serum samples (from approximately 70 μl blood) were collected weekly for ELISA analysis until sacrifice by cardiac puncture at day 50.

TABLE 3
Mice used in experiment 3 (I.P.2)

| Genotype | Immunisation | n= |
|---|---|---|
| WT (C57BL/6) | 2.7 μgSbi-III-IV-Ag85b in 200 μl 150 mM | 4 |
| | NaCl (Conjugate) | |
| WT (C57BL/6) | 0.7 μgSbi-III-IV plus 2 μg Ag85b in | 4 |
| | 200 μl 150 mM NaCl (Separate) | |
| WT (C57BL/6) | 2 μg Ag85b in 200 μl 150 mM NaCl | 2 |
| C3^{-/-} (C57BL/6) | 2.7 μg Sbi-III-IV-Ag85b in 200 μl | 4 |
| | 150 mM NaCl (Conjugate) | |
| C3^{-/-} (C57BL/6) | 0.7 μgSbi-III-IV plus 2 μg Ag85b in | 4 |
| | 200 μl 150 mM NaCl (Separate) | |
| C3^{-/-} (C57BL/6) | 2 μg Ag85b in 200 μl 150 mM NaCl | 2 |

### ELISA Assays to Test Immune Response to Ag85b

96 well plates (NUNC Maxisorb) were coated with 1 μg/ml Ag85b or 1.35 μg/ml Sbi-III-IV-Ag85b in carbonate buffer at 50 μl per well and incubated at 4° C. overnight. Plates were washed with 0.01% PBS-Tween and a 1% BSA blocking solution was incubated for 1 hour at room temperature and then washed. Serum samples were diluted to 1/50 or 1/100 in 0.01% PBS-Tween, added at 50 μl per well and incubated for 1 hour at room temperature. Plates were washed and secondary antibody (sheep anti-mouse IgG HRPO) was added at 1/100 dilution at 50 μl per well and incubated for 1 hour at room temperature. TMB substrate was prepared (200 μl TMB in DMSO (10 mg/ml), 9.9 ml phosphate-citrate buffer, 3 μl H₂O₂). Plates were washed and 50 μl per well TMB substrate was added and allowed to develop for 6 minutes. The reaction was stopped with 50 μl per well 10% H₂SO₄ and plates were read at 450 nm.

This was repeated for each serum sample, collected weekly.

For normalisation purposes, an average baseline ‘Day 0’ value was calculated for the entire group of mice and separate ‘Day 0’ average values calculated for each individual treatment group. During the course of the study, each data point was normalised to the global ‘Day 0’ average of all mice divided by the ‘Day 0’ OD450 reading for the relevant treatment group.

### Effect of Sbi-III-IV on Th1 Response Against Ag85b

Male mice were injected intraperitoneally with molar equivalent amounts of either Sbi (n=2), Ag85b (n=3), Sbi-Ag85b (n=3) diluted in PBS, PBS alone (n=3), or Ag85b diluted in the commonly used adjuvant Alum (n=2). See FIG. 9A for a schematic outline of the experiment. All volumes injected were 200 μl. Experiments were carried out in two blocks. 5 days after injection, mice were euthanised and spleens removed. Spleens were processed for in vitro splenocyte cultures, and added to 6 well plates (2×10⁶ cells/well) containing dendritic cells (1×10⁶ cells/well; clone DC2.4) that had been primed with 1 μg/well of Ag85b 24 hours previously. Cultures were incubated for 72 hrs and supernatant collected for analysis in cytokine specific ELISAs according to manufacturer's instructions (R&D systems). Cells were then treated with 50 ng of PMA (Calbiochem), 500 ng Ionomycin (Sigma) and 1 μl of BD Golgi block (BD Biosciences) in fresh media. After a 5 hour incubation period (37° C., 5% CO₂) cells were pelleted, washed in PBS and resuspended in 2.4G2 Fc block (BD Pharmingen) and heat inactivated mouse Ig (both 1/100 in PBS) for 30 minutes on ice.

Live (Aqua live dead negative) T-Helper cells (Th) were identified using forward and side scatter (singlets) in conjunction with positive reaction with CD3-PE and CD4-PerCP^{Cy5.5} antibodies (and negative for CD8-APC^{H7}) using standard flow cytometry analysis i.e. antibodies were added to each well of a 96 well ELISA plate as required at 1:200 diluted in 25 μl flow buffer. 100 μl of blocked cells were added to each well and incubated on ice in the dark for 30 minutes. Cells were then fixed in 4% formaldehyde solution for 20 minutes. To stain for intracellular proteins cells were permeabilised in 100 μl of BD Perm (BD Biosciences, 15 minute incubation on ice), then incubated with intracellular antibodies (IFN-γ-PE^{Cy7} or IgG_{2b}-APC Isotype control) on ice in the dark for 30 minutes. Finally, cells were resuspended in 250 μl of flow buffer and read on the FACS CANTO II machine (BD Biosciences). Flow cytometry data was analysed using FCS express 6 Flow Research Edition software (DeNovo Software). All data is expressed as a percentage relative to numbers of T cells in the PBS control culture and statistical analysis was carried out on GraphPad Prism 7.0.

Th1 proliferation stimulated by the SbiIII-IV-Ag85b conjugate was significantly higher than that stimulated by Ag85b alone or in combination with alum (FIG. 9C). Secreted IFN-γ in the tissue culture supernatant of splenocytes primed with Sbi-III-IV-Ag85b was increased by 4 fold compared to cells primed with Ag85b alone (FIG. 9D).

### Effect of Sbi-III-IV on Dendritic Cell Activation and Function

3×10⁶ healthy donor PBMC, separated by standard density centrifugation, were cultured in RPMI plus 50% autologous serum, in the presence or absence of Poly(I:C) (10 μg/ml, Invivogen), Lipopolysaccharide (LPS, 10 ng/ml, Sigma), CL075 (1 μg/ml, Invivogen) and CpG (ODN 2216, 7.5 μg, Invivogen), with or without SBI (10 μg/ml; confirmed endotoxin free). Cells were cultured for 14 hrs at 37° C., 5% CO₂, with addition of Brefeldin A (10 μg/ml, eBioscience) after 3 hrs. Cells were stained with Zombie amine dye (Biolegend) for dead cell exclusion (usually <30%), surface markers and then intracellular cytokines after fixation and permeabilization (eBioscience) according to manufacturer's protocol. Analysis was performed with an LSRFortessa X-20 running BD FACSDIVA™ 8.0.1 software and analysed with FlowJo 10.1r5 (Tree Star, Inc). Graphs were plotted with Prism V5 (GraphPad software Inc).

The lineage⁻ (CD3,16,19,20) HLA-DR⁺ population was identified from within the live, singlet cells. This fraction contained CD14⁺ monocytes as well as CD14⁻ dendritic cell (DC) populations. The CD14⁻ DC were subdivided into CD123⁺ plasmacytoid dendritic cells (pDC) and the CD123⁻ CD141⁺ (cDC1) or CD2⁺CD1c⁺CD11c⁺ (cDC2) classical dendritic cells. Cytokine production was quantified as the percentage of positive cells/parent population.

Antibodies were from Biolegend (Bio) or BD Biosciences (BD) unless otherwise stated, denoted as antigen-fluorochrome, clone (manufacturer): CD11c-BV711, B-ly6 (BD); CD123-BUV395, 7G3 (BD); CD14-BV650, M5E2 (Bio), CD141-BV510, 1A4 (BD); CD19-AF700, H1B1 (Bio); CD20-AF700, 2H7 (Bio); CD3-AF700, SK7 (Bio); CD16-AF700, 3G8 (Bio); CD1c-PERCP-Cy5.5, L161 (Bio); CD303-BV605, 201A (Bio); CD304-BV605, U21-1283 (BD); HLA0DR-BV780, L243 (Bio); IFNa-PE, LT27:295 (MACS Miltenyi Biotec); IL-10-APC, JES3-9D7 (Bio); IL-12p40/p70-BV421, C8.6 (BD); IL-1b-FITC, JK1B-1 (Bio); IL-8-PE-Cy7, E8N1 (Bio); TNF-APCCy7, Mabll (Bio).

The adjuvant effect of Sbi-III-IV was investigated by examination of intracellular cytokine production by specific monocyte and dendritic cell subsets in response to a cocktail of TLR agonists (Poly(I:C), Lipopolysaccharide, CL075 and CpG). Culture in the presence of 10 μg/ml of Sbi-III-IV increased the percent of cells producing TNFα in all cell subsets examined, both with and without the addition of TLR agonist cocktail. In the presence of TLR agonists, Sbi-III-IV potentiated monocyte production of IL-1b and IFNα elaboration from plasmacytoid DCs. IL-10 production from monocytes was reduced, thus demonstrating an adjuvant action of Sbi-III-IV on human primary immune effector cells as evidenced by an increase in the production of inflammatory cytokines and reduction of anti-inflammatory IL-10 from monocytes. INFO production from all cells and IFNα production from plasmacytoid DCs was also increased, independently of TLR agonist stimulation.

### Synthesis of Sbi-III-IV(V80C)-Pn6B Conjugate

The antigenic polysaccharide Pn6B from the outer protective capsule of the pathogen Streptococcus pneumoniae was chosen as a ligand for functionalisation to Sbi-III-IV protein, as it is a common antigen utilised in vaccine production to prevent infection from S. pneumoniae. Pn6B is a 0.9-1.5 MDa carbohydrate and consists of a repeat unit of (→2-α-D-Galactopyranose-(1→3)-α-D-Glucopyranose-(1→3)-α-L-rhamnopyranose-(1→4)-D-ribitol-5-phosphate→).

Pn6B (20 mg) was added to 2 mL of water and stirred at room temperature until the carbohydrate had dissolved (3-6 hours). The solution was then loaded onto an ion exchange column (Dowex 50W×4-200, tetrabutylammonium form) and incubated for 30 minutes before allowing elution. The fractions containing carbohydrate were pooled and subsequently freeze dried to yield a white solid. The solid was added to 5 mL of anhydrous DMSO and stirred at 30° C. under N2 overnight to dissolve the carbohydrate. An anhydrous DMSO solution (1 mL) of 1,1′-carbonyl diimidazole (1-2 mg) was added to the carbohydrate solution and allowed to stir for one hour. Triethylamine (0.05 mL) was added, followed by N-(2-(2-(2-aminoethoxy)ethoxy)-ethyl)-3-(2,5-dioxo-2,5-dihydro-1H-pyrrol-1-yl)propanamide (100 mg), and the reaction was left to stir for 2 hours. The solution was transferred to a dialysis bag (12-14 KDa cut-off) and dialysed against 1) 5 litres of 0.1 M sodium phosphate buffer (pH=7), 2) 5 litres of 0.01 M sodium phosphate buffer (pH=7), 3) 5 litres of water. The solution in the dialysis bag was then treated with ion exchange resin (Dowex 50W×8-100, H+ form). The resin was removed by centrifugation. The supernatant was freeze dried to yield 24 mg of white solid. A portion of product was analysed by NMR spectroscopy to determine alkylating reagent loading on the carbohydrate. This was determined through the comparison of the integrals of the maleimide's alkene proton signal and the fucose's methane proton signals.

Sbi-III-IV(V80C) (0.5 mg, 3.57×10-2 mmol) was dissolved in deoxygenated sodium phosphate buffer (0.5 mL, 200 mM, pH=7, containing 1 mM EDTA and 5 eq. of THPP). The reactions were incubated at 25° C. for 24 hours. 5-Azido pentanoic acid (5 eq.) was subsequently added to oxidise the remaining phosphine in solution and incubated for 30 minutes. Finally, a deoxygenated solution (0.5 ml, water) of Pn6B-maleimide (0.5 mg) was added to the freshly reduced protein and incubated at ambient temperature for up to 24 hours. SDS PAGE was used to analyse the bio conjugation reaction. Aliquots of the solution (10 μL) were added to reducing SDS PAGE loading buffer (10 μL). A sample (10 μL) of this solution was loaded into a precast gel for SDS PAGE electrophoresis (Invitrogen, NuPage 4-12% Bis-Tris gradient gel, NuPage MOPS SDS running buffer, Bio Rad PowerPac HV, 200 V, 45-55 minutes). The gel was subsequently stained with Coomassie stain, followed by soaking in a destaining solution (water:ethanol:acetic acid, 16:3:1). The presence of stained material within the loading wells of the gel provides a positive indication of a high molecular weight carbohydrate-protein conjugate. Size exclusion high performance liquid chromatography (SEC-HPLC) was also utilised to confirm conjugate production. A sample (10 μL) of the reaction was injected into a Dionex Ultimate 3000 HPLC instrument equipped with a TOSOH TSKgel SEC-column (G5000PWXL, 7.8 mm I.D.×30.0 cm L). The sample was eluted at 0.5 ml/min with sodium phosphate buffer (50 mM, pH=7), containing sodium chloride (150 mM), with the detector set at 280 nm. The conjugate is represented by a broad peak, eluting from 12-20 minutes, while the unreacted Sbi protein elutes at 23-24 minutes.

The conjugation reaction was quenched by the addition of cysteamine (5 mM) and the unreacted Sbi protein in the reaction mixture was removed spin filter centrifugation (30 kDa cut-off membrane).

### In Vitro Complement Activation by Sbi-III-IV(V80C)-Pn6B

The Wielisa total complement system screen (Wieslab), described by Seelen et al. was used to detect depletion by Sbi-III-IV, Pn6B and Sbi-III-IV(V80C)-Pn6B of the classical (CP), mannose-binding lectin (MBLP), and alternative (AP) complement pathways. 1 μg of Sbi or the Sbi conjugate was added per 1 μl of human serum (positive control serum, supplied with the kit), and assayed for complement activity after 30 min of preincubation at 37° C. The assay was completed in duplicate, according to the manufacturer's instructions, and included a blank, a positive control (human serum from healthy individuals), and a negative control (heat-inactivated serum). Residual complement activity inhibition (i.e. complement activity remaining after depletion) was quantified from the absorbance at 405 nm using the equation: (sample−negative control)/(positive control−negative control)×100%.

### T Cell Activation by Exposure to Sbi-III-IV(V80C)-Pn6B

Peripheral blood donated by a healthy human volunteer was heparinised (10 Units/ml) in syringes and mixed at a 1:1 ratio with RPMI 1640 medium. 35 ml of blood/RPMI was layered on top of 15 ml Lymphoprep (Greiner Bio One) in 50 ml Falcon tubes. These were centrifuged for 30 minutes at 1500 RPM with the brake off on deceleration. The Peripheral blood mononuclear cell (PBMC) layer was then extracted from the tube using a Pasteur pipette and washed three times in RPMI 1640 medium. PBMCs were re-suspended in RPMI medium+10% human serum with complement. PBMC's were then treated with 200 μl of Sbi-III-IV or the Sbi-III-IV(V80C)-Pn6B conjugate protein (10 μg/ml), then incubated at 37° C. (5% CO2) for 24 hours. Cells were then washed once in cold PBS, suspended in 100 μl PBS and then treated with 10 μl labelled antibody (anti-CD69). The antibody labelled cells were then incubation at 4° C. for 30 minutes, then washed twice in ice cold PBS, re-suspended in 400 μl PBS and analysed using a FACSCanto flow cytometer (BD Bioscience) and processed using DIVA software.

CD69 is an important indicator of T cell activation. Exposure of lymphocytes to the Sbi-III-IV(V80C)-Pn6B conjugate resulted in a significant increase in surface expression of CD69 compared to unconjugated Pn6B antigen, indicating enhanced T cell response against the conjugate compared to the unconjugated antigen (data not shown).

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

1. A complement-activating moiety comprising Sbi-III-IV for use as an immunological adjuvant.

2. A complement-activating moiety comprising Sbi-III-IV for use in a method of enhancing an immune response in a subject against a target antigen, wherein said complement-activating moiety is administered to a subject in conjunction with the target antigen.

3. A complement-activating moiety for use according to claim 2 wherein the target antigen is a peptide antigen.

4. A complement-activating moiety for use according to claim 2 wherein the target antigen comprises a carbohydrate, saccharide, polysaccharide, lipid or lipopolysaccharide.

5. A complement-activating moiety for use according to any one of claims 2 to 4 wherein the target antigen is admixed with the complement-activating moiety.

6. A complement-activating moiety for use according to claim 2 or claim 3 wherein the target antigen is covalently linked to the complement-activating moiety.

7. A complement-activating moiety for use according to claim 6 wherein the target antigen forms a fusion protein with the complement-activating moiety.

8. A complement-activating moiety for use according to claim 4 wherein the target antigen is covalently linked to a carrier peptide.

9. A method of enhancing the immunogenicity of a target antigen, comprising contacting the target antigen in vitro or ex vivo with complement and a complement-activating moiety comprising Sbi-III-IV to yield an opsonised target antigen.

10. A method according to claim 9, further comprising the step of isolating the opsonised target antigen from other complement components and/or the complement activating moiety.

11. A method according to claim 9 or claim 10, further comprising the step of formulating the opsonised target antigen for administration to a subject.

12. A method according to claim 9 or claim 10 further comprising administering the target antigen to a subject.

13. A composition comprising an opsonised target antigen for use in a method of stimulating an immune response against the target antigen, wherein the target antigen has previously been contacted in vitro or ex vivo with complement and a complement-activating moiety comprising Sbi-III-IV.

14. A complement-activating moiety for use according to any one of claims 2 to 8, a method according to any one of claims 10 to 12, or a composition for use according to claim 13, wherein the target antigen is derived from an infectious organism.

15. A complement-activating moiety for use, a method, or a composition for use, according to claim 14, wherein the infectious organism is a bacterium, fungal cell, virus, protozoan, helminth or fluke.

16. A complement-activating moiety for use, a method, or a composition for use, according to claim 15, wherein the bacterium is:
Actinomyces (e.g. Actinomyces israelii);
Bacillus (e.g. Bacillus anthracis, Bacillus cereus);
Bacteroides (e.g. Bacteroides fragilis);
Bartonella (e.g. Bartonella henselae, Bartonella quintana);
Bordetella (e.g. Bordetella pertussis);
Borrelia (e.g. Borrelia burgdorferi, Borrelia garinii, Borrelia afzelii, Borrelia recurrentis);
Brucella (e.g. Brucella abortus, Brucella canis, Brucella melitensis, Brucella suis);
Campylobacter (e.g. Campylobacter jejuni);
Chlamydia and Chlamydophila (e.g. Chlamydia pneumoniae Chlamydia trachomatis Chlamydophila psittaci);
Clostridium (e.g. Clostridium botulinum, Clostridium difficile, Clostridium perfringens Clostridium tetani);
Corynebacterium (e.g. Corynebacterium diphtheriae);
Cryptococcus (e.g. Cryptococcus neoformans);
Ehrlichia (e.g. Ehrlichia canis, Ehrlichia chaffensis);
Enterococcus (e.g. Enterococcus faecalis, Enterococcus faecium);
Escherichia (e.g. Escherichia coli);
Francisella (e.g. Francisella tularensis);
Haemophilus (e.g. Haemophilus influenzae);
Helicobacter (e.g. Helicobacter pylori);
Klebsiella (e.g. Klebsiella pneumoniae);
Legionella (e.g. Legionella pneumophila);
Leptospira (e.g. Leptospira interrogans, Leptospira santarosai, Leptospira weilii, Leptospira noguchii);
Listeria (e.g. Listeria monocytogenes);
Mycobacterium (e.g. Mycobacterium leprae, Mycobacterium tuberculosis, Mycobacterium ulcerans);
Mycoplasma (e.g. Mycoplasma pneumoniae);
Neisseria (e.g. Neisseria gonorrhoeae, Neisseria meningitidis);
Nocardia (e.g. Nocardia asteroides);
Pseudomonas (e.g. Pseudomonas aeruginosa);
Rickettsia (e.g. Rickettsia rickettsii);
Salmonella (e.g. Salmonella typhi, Salmonella typhimurium, Salmonella enterica);
Shigella (e.g. Shigella sonnei, Shigella dysenteriae; Shigella flexneri);
Staphylococcus (e.g. Staphylococcus aureus, Staphylococcus epidermidis, Staphylococcus saprophyticus);
Streptococcus (e.g. Streptococcus agalactiae, Streptococcus pneumoniae, Streptococcus pyogenes, Streptococcus viridans);
Treponema (e.g. Treponema pallidum);
Ureaplasma (e.g. Ureaplasma urealyticum);
Vibrio (e.g. Vibrio cholerae); or
Yersinia (e.g. Yersinia pestis, Yersinia enterocolitica, Yersinia pseudotuberculosis).

17. A complement-activating moiety for use, a method, or a composition for use, according to claim 15, wherein the fungal cell is:
Candida (e.g. Candida albicans, Candida glabrata, Candida rugosa, Candida parapsilosis, Candida tropicalis, Candida dubliniensis);
Aspergillus (e.g. Aspergillus fumigatus, Aspergillus flavus);
Cryptococcus (e.g. Cryptococcus neoformans);
Histoplasma (e.g. Histoplasma capsulatum);
Pneumocystis (e.g. Pneumocystis jirovecii, Pneumocystis carinii); or
Stachybotrys (e.g. Stachybotrys charatum)

18. A complement-activating moiety for use, a method, or a composition for use, according to claim 15, wherein the virus is of the type:
Adenoviridae (e.g. Adenovirus);
Herpesviridae (e.g. Herpes simplex, type 1, Herpes simplex, type 2, Varicella-zoster virus, Epstein-Barr virus, Human cytomegalovirus, Human herpesvirus type 8);
Papillomaviridae (e.g. Human papillomavirus);
Polyomaviridae (e.g. BK virus, JC virus);
Poxviridae (e.g. Smallpox);
Hepadnaviridae (e.g. Hepatitis B virus);
Parvoviridae (e.g. Parvovirus B19);
Astroviridae (e.g. Human astrovirus);
Caliciviridae (e.g. Norwalk virus);
Picornaviridae (e.g. coxsackievirus, hepatitis A virus, poliovirus, rhinovirus);
Coronaviridae (e.g. Severe acute respiratory syndrome virus);
Flaviviridae (e.g. Hepatitis C virus, yellow fever virus, dengue virus, West Nile virus, TBE virus);
Togaviridae (e.g. Rubella virus);
Hepeviridae (e.g. Hepatitis E virus);
Retroviridae (e.g. Human immunodeficiency virus (HIV), Human T-cell leukaemia virus (HTLV) types I, II, III and IV);
Orthomyxoviridae (e.g. Influenza virus);
Arenaviridae (e.g. Lassa virus);
Bunyaviridae (e.g. Crimean-Congo hemorrhagic fever virus, Hantaan virus);
Filoviridae (e.g. Ebola virus, Marburg virus);
Paramyxoviridae (e.g. Measles virus, Mumps virus, Parainfluenza virus, Respiratory syncytial virus);
Rhabdoviridae (e.g. Rabies virus);
Hepatitis D virus;
Reoviridae (e.g. Rotavirus, Orbivirus, Coltivirus, Banna virus).

19. A complement-activating moiety for use, a method, or a composition for use, according to claim 15, wherein the protozoan is:
Plasmodium spp. (responsible for malaria, e.g. Plasmodium falciparum, Plasmodium berghei, Plasmodium yoelii, Plasmodium vivax and Plasmodium knowlesii)
Entamoeba (Entamoeba histolytica is responsible for amoebic dysentery);
Giardia (responsible for Giardiasis, e.g. Giardia lamblia);
trypanosomes (e.g. Trypanosoma brucei, which causes African sleeping sickness, and Trypanosoma cruzi);
Leishmania (e.g. Leishmania spp. and Leishmania mexicana);
Toxoplasma (e.g. Toxoplasma gondii);
Acanthamoeba;
Babesia;
Balamuthia (e.g. Balamuthia mandrillaris)
Cryptosporidium;
Cyclospora;
Naegleria (e.g. Naegleria fowleri).

20. A complement-activating moiety for use, a method, or a composition for use, according to any one of claims 2 to 15 wherein the target antigen is a marker expressed specifically or preferentially on a neoplastic cell.

21. A complement-activating moiety for use, a method, or a composition for use, according to any one of the preceding claims, wherein said complement-activating moiety does not bind immunoglobulin Fc.

22. A complement-activating moiety for use, a method, or a composition for use, according to any one of the preceding claims wherein said complement-activating moiety does not comprise Sbi-I or Sbi-II.

23. A complement-activating moiety for use, a method, or a composition for use, according to any one of the preceding claims, wherein the complement-activating moiety comprises an Sbi-III domain with at least 80%, at least 85%, at least 90%, at least 95% or at least 99% sequence identity with the wild type Sbi-III sequence.

24. A complement-activating moiety for use, a method, or a composition for use, according to any one of the preceding claims, wherein the complement-activating moiety comprises an Sbi-IV domain with at least 80%, at least 85%, at least 90%, at least 95% or at least 99% sequence identity with the wild type Sbi-IV sequence.

25. A complement-activating moiety for use, a method, or a composition for use, according to any one of the preceding claims, wherein the complement-activating moiety comprises an Sbi-III-IV moiety with at least 80%, at least 85%, at least 90%, at least 95% or at least 99% sequence identity with the wild type Sbi-III-IV sequence.

26. A method of enhancing the immunogenicity of a target antigen, comprising associating said target antigen with a complement-activating moiety comprising Sbi-III-IV.

27. A method according to claim 22 wherein the target antigen is associated with the complement-activating moiety by:
(i) admixing the target antigen with the complement-activating moiety;
(ii) covalently linking the target antigen to the complement-activating moiety; or
(iii) expressing the target antigen as a fusion protein with the complement-activating moiety.

28. A method of immune stimulation, comprising administering a complement-activating moiety comprising Sbi-III-IV as an immunological adjuvant.

29. A method of enhancing an immune response in a subject against a target antigen, wherein said method comprises administering a complement-activating moiety comprising Sbi-III-IV to the subject in conjunction with the target antigen.

30. Use of a complement-activating moiety comprising Sbi-III-IV in the preparation of a medicament for use as an immunological adjuvant.

31. Use of a complement-activating moiety comprising Sbi-III-IV in the preparation of a medicament for use in a method of enhancing an immune response in a subject against a target antigen, wherein said method comprises administering the complement-activating moiety to the subject in conjunction with the target antigen.

32. A method of stimulating an immune response against a target antigen, wherein the method comprises administering to the subject a target antigen which has previously been contacted in vitro or ex vivo with complement and a complement-activating moiety comprising Sbi-III-IV.

33. Use of a composition comprising an opsonised target antigen in the preparation of a medicament for stimulating an immune response against the target antigen, wherein the target antigen has previously been contacted in vitro or ex vivo with complement and a complement-activating moiety comprising Sbi-III-IV.
